SEQUENCE LISTING (6 sequences)

SEQ ID NO: 1 - DNA, 19 bp, Artificial Sequence
  Synthetic Primer Flap A
cccaagaacccaaggaaca

SEQ ID NO: 2 - DNA, 25 bp, Artificial Sequence
  Synthetic Primer Flap S
agacaagatagaggaagagcaaaac

SEQ ID NO: 3 - DNA, 27 bp, Artificial Sequence
  Synthetic Lenti TM probe
aaccattaggagtagcacccaccaagg

SEQ ID NO: 4 - DNA, 24 bp, Artificial Sequence
  Synthetic Actine A primer
cggtgaggatcttcatgaggtagt

SEQ ID NO: 5 - DNA, 20 bp, Artificial Sequence
  Synthetic Actine S primer
aacaccccagccatgtacgt

SEQ ID NO: 6 - DNA, 22 bp, Artificial Sequence
  Synthetic HUMURA ACT TM probe
ccagccaggtccagacgcagga

(19) United States
(12) Patent Application Publication
(10) Pub. No.: US 2017/0000849 A1
(43) Pub. Date: Jan. 5, 2017

(54) CD300A RECEPTORS AS VIRUS ENTRY COFACTORS

(71) Applicants: INSTITUT NATIONAL DE LA SANTE ET DE LA RECHERCHE MEDICALE (INSERM), Paris (FR); CENTRE NATIONAL DE LA RECHERCHE SCIENTIFIQUE (C.N.R.S.), Paris (FR); UNIVERSITE PARIS DIDEROT - PARIS 7, Paris (FR)

(72) Inventors: Ali AMARA, Paris (FR); Xavier CARNEC, Paris (FR); Laurent MEERTENS, Paris (FR)

(21) Appl. No.: 15/106,467

(22) PCT Filed: Dec. 19, 2014
(86) PCT No.: PCT/EP2014/078893

§ 371 Date: Jun. 20, 2016

(30) Foreign Application Priority Data
Dec. 20, 2013 (EP) 13306820.5

(51) Int. Cl. A61K 38/17; C07K 16/28; C12N 15/113; C07K 16/10; A61K 39/42; A61K 38/12; A61K 45/06; A61K 39/395

(52) U.S. Cl. CPC A61K 38/1774; A61K 45/06; C07K 16/2803; A61K 39/3955; C07K 16/1081; A61K 39/42; A61K 38/1709; A61K 38/12; C12N 15/1138; C12N 2310/11; C07K 2317/76; C12N 2320/30

(57) ABSTRACT

The present invention concerns an inhibitor of an interaction between CD300a and an aminophospholipid for use in preventing or treating a virus infection by inhibition of the interaction between CD300a and viral aminophospholipid.

The present invention concerns the use of an inhibitor of an interaction between CD300a and an aminophospholipid for preventing or treating a viral infection.

### BACKGROUND TO THE INVENTION

Viral infections are a major threat to public health. The emergence and expansion of life-threatening diseases caused by viruses (e.g. hemorrhagic fever and encephalitis), together with unmet conventional prevention approaches (e.g., vaccines) highlights the necessity of exploring new strategies that target these deadly pathogens.

The Flavivirus genus for example encompasses over 70 small-enveloped viruses containing a single positive-stranded RNA genome. Several members of this genus such as Dengue virus (DV), Yellow Fever Virus (YFV), and West Nile virus (WNV), are mosquito-borne human pathogens causing a variety of medically relevant human diseases including hemorrhagic fever and encephalitis (Gould and Solomon, 2008, Lancet, 371:200-509; Gubler et al., 2007, Fields Virology, 5^{th} Edition, 1153-1252). Dengue disease, which is caused by four antigenically related serotypes (DV1 to DV4), has emerged as a global health problem during the last decades and is one of the most medically relevant arboviral diseases. It is estimated that 50-100 million dengue cases occur annually and more than 2.5 billion people are at risk of infection. Infection by any of the four serotypes causes diseases, ranging from mild fever to life-threatening dengue hemorrhagic fever (DHF) and dengue shock syndrome (DSS). Despite the importance and increasing incidence of DV as a human pathogen, there is currently no licensed vaccine available against DV and the lack of anti-viral drugs severely restricts therapeutic options.

Future efforts to combat dengue disease require a better understanding of the DV life cycle. DV entry into target cells is a promising target for preventive as well as therapeutic anti-viral strategies since it is a major determinant of the host-range, cellular tropism and viral pathogenesis. During primary infection, DV enters host cells by clathrin-mediated endocytosis, a process driven by the interaction between the viral glycoprotein (E protein) with cellular receptors. Within the endosome, the acidic environment triggers an irreversible trimerization of the E protein that results in fusion of the viral and cell membranes, allowing the release of the viral capsid and genomic RNA into the cytosol. To date, the molecular bases of DV-host interactions leading to virus entry are poorly understood and little is known about the identity of the DV cellular receptor(s). DV is known to infect a wide range of cell types. DV may thus exploit different receptors, depending on the target cell, or use widely expressed entry molecules. Earlier studies indicated that DV virions make initial contact with the host by binding to heparan-sulfate proteoglycans on the cell membrane. These molecules recognize the positively charged residues on the surface of E protein and are thought to concentrate the virus at the target cell surface before its interactions with entry factors. Numerous cellular proteins such as heat shock protein 70 (HSP70), HSP90, GRP78/Bip, a lipopolysaccharide receptor-CD14 or the 37/67 kDa high affinity laminin have been proposed as putative DV entry receptors.

However, their function in viral entry remains poorly characterized and of unclear physiological relevance. To date, the only well-characterized factors that actively participate in the DV entry program are DC-SIGN expressed on dendritic cells, L-SIGN expressed on liver sinusoidal endothelial cells and the mannose receptor (MR) expressed on macrophages. These molecules belong to the C-type lectin receptor family and bind mannose-rich N-linked glycans expressed on the DV E protein.

Recently the inventors discovered that TIM and TAM proteins, proteins of two receptor families that mediate the phosphatidylserine (PtdSer)-dependent phagocytic removal of apoptotic cells, serve as DV entry factors.

However, DV infects cell types that do not express DC-SIGN, MR, L-SIGN, or TIM and TAM which thus indicates that other relevant entry receptor(s) and/or entry mechanisms exist and remain to be identified.

Furthermore, DV has become a global problem and is endemic in more than 110 countries. Thus, development of a prophylactic or curative treatment of DV infection is needed.

Moreover, deciphering the mechanism of DV internalization might also pave the way to developing treatment of other viral infections.

### DESCRIPTION OF THE INVENTION

The inventors have found that DV infection is mediated by the interaction between phosphatidylserine (PtdSer) and/or phosphatidylethanolamine (PtdEth) at the surface of the DV viral envelope and the receptor CD300a present at the surface of the host cell, and that such interaction can be blocked, thereby inhibiting entry of DV into host cells and preventing DV infection.

The inventors showed that blocking the interaction between receptor CD300a present at the surface of those host cells and phosphatidylserine and/or phosphatidylethanolamine thereby inhibiting entry of DV into host cells and preventing DV infection is a new therapeutic approach to inhibit antibody-dependent enhancement of DV infections.

Furthermore, the inventors found that this interaction between aminophospholipids such as phosphatidylserine (PtdSer) and/or phosphatidylethanolamine (PtdEth) and receptor CD300a is also used by other aminophospholipid harboring viruses, in particular aminophospholipids harboring flaviviruses.

This interaction thus represents a general mechanism exploited by viruses that incorporate aminophospholipids, in particular phosphatidylserine (PtdSer) and/or phosphatidylethanolamine (PtdEth) in their membrane.

The invention thus relates to an inhibitor of an interaction between CD300a and an aminophospholipid for use in preventing or treating a virus infection by inhibition of the interaction between CD300a and a viral aminophospholipid.

The virus may be an aminophospholipid harboring virus, in particular an aminophospholipid harboring Flavivirus, in particular a phosphatidylserine harboring Flavivirus and/or phosphatidylethanolamine harboring Flavivirus.

Preferably, aminophospholipid harboring Flavivirus are West-Nile Virus, Yellow Fever Virus or Dengue Virus.

Preferably, the aminophospholipid is phosphatidylserine (PtdSer) and/or phosphatidylethanolamine (PtdEth).

Preferably, the inhibitor according to the invention is for use in preventing or treating a virus infection in myeloid cells dendritic cells, mast cells, granulocytes and/or monocytes, more particular monocytes and/or mast cells.

Preferably, the inhibitor according to the invention is for use in preventing or treating a virus infection, preferably an aminophospholipid harboring virus infection, in a subject that is at risk of suffering from antibody-dependent enhancement of infection.

Preferably, said inhibitor of an interaction between CD300a and an aminophospholipid is a CD300a inhibitor and/or an aminophospholipid binding protein.

Preferably, said CD300a inhibitor is an anti-CD300a antibody, an antisense nucleic acid, a mimetic or a variant CD300a.

Preferably, said aminophospholipid binding protein is a phosphatidylserine binding protein and/or phosphatidylethanolamine binding protein.

Preferably, the phosphatidylserine binding protein is an anti-phosphatidylserine antibody or Annexin 5.

Preferably, the phosphatidylethanolamine binding protein is an anti-phosphatidylethanolamine antibody or Duramycin.

Further provided is the use of an inhibitor of an interaction between aminophospholipids and receptor CD300a in a method of inhibiting entry of a virus, in particular an aminophospholipid harboring virus such as an aminophospholipid harboring flavivirus, into a cell.

Also provided is a method for preventing or treating a viral infection, in particular an aminophospholipid harboring virus infection such as an aminophopsholipid harboring flavivirus infection, comprising administering to an individual in need thereof a therapeutically effective amount of an inhibitor of an interaction between aminophospholipid and receptor CD300a.

Also provided is the use of an inhibitor of an interaction between an aminophospholipid and receptor CD300a for the manufacture of a medicament for preventing or treating a viral infection, in particular an aminophospholipid harboring virus infection, in particular an aminophospholipid harboring flavivirus infection.

Further the inhibitor is for administration in combination with at least one other antiviral compound, either sequentially or simultaneously.

### DEFINITIONS

By “an aminophospholipid harboring virus” is meant an enveloped virus that expresses or incorporates aminophospholipids, in particular PtdSer and/or PtdEth in its membrane. An aminophospholipid harboring virus may be in particular an aminophospholipid harboring Flavivirus, in particular a phosphatidylserine harboring Flavivirus and/or phosphatidylethanolamine harboring Flavivirus.

Preferably, aminophospholipid harboring Flavivirus are West-Nile Virus, Yellow Fever Virus or Dengue Virus.

Throughout the instant application, the term “and/or” is a grammatical conjunction that is to be interpreted as encompassing that one or more of the cases it connects may occur. For example, the wording “aminophospholipid is phosphatidylserine and/or phosphatidylethanolamine” indicates that the aminophospholipid in context of the invention may be phosphatidylserine or phosphatidylethanolamine or phosphatidylserine and phosphatidylethanolamine.

By “an aminophospholipid harboring virus infection” is thus meant an infection with an enveloped virus that expresses or incorporates aminophospholipids, in particular PtdSer and/or PtdEth in its membrane. Prior to infection, the aminophospholipids, in particular PtdSer and PtdEth, are exposed on the viral membrane to receptors of the host cell, in particular to the CD300a receptor of the host cell. An aminophospholipid harboring virus infection may include, for example, an “aminophospholipid harboring flavivirus infection”. By “aminophospholipid harboring flavivirus infection” is meant an infection with for example Dengue virus (DV), a West Nile virus or a yellow fever virus. The Dengue virus may be of any serotype, i.e. serotype 1, 2, 3 or 4.

By “interaction between CD300a and an aminophospholipid” is meant the direct interaction between the receptor CD300a present at the surface of the host cell and an aminophospholipid, in particular PtdSer and/or PtdEth, present at the surface of the virus of the aminophospholipid harboring virus. In fact, the inventors have found that the direct interaction between CD300a and an aminophospholipid such as PtdEth and/or PtdSer permits the aminophospholipid harboring virus infection or entry into the host cells.

In the context of the invention, “antibody-dependent enhancement of infection” refers to a mechanism of infection that occurs when preexisting antibodies present in the body from a primary (first) virus infection bind to an infecting virus-particle during a subsequent infection with a different virus serotype. The antibodies from the primary infection cannot neutralize the virus. Instead, the Antibody-virus complex attaches to receptors called Fcγ receptors (FcγR) on circulating monocytes. The antibodies help the virus infecting monocytes more efficiently. The outcome is an increase in the overall replication of the virus and a higher risk of a severe virus infection.

CD300a is expressed in myeloid cells for example dendritic cells, mast cells, granulocytes and/or monocytes.

Thus, in a preferable embodiment, the inhibitor according to the invention is for use for preventing or treating a virus infection in myeloid cells, in particular dendritic cells, mast cells, granulocytes and/or monocytes, more particular monocytes and/or mast cells. As described above monocytes are sensitive to Antibody-dependent enhancement (ADE). Thus, in a particular embodiment, the inhibitor according to the invention is for use in preventing or treating a virus infection, preferably an aminophospholipid harboring virus infection, in a subject that is at risk of suffering from antibody-dependent enhancement of infection.

Preferably, the subject that is at risk of suffering from antibody-dependent enhancement of infection has already been infected, at least once, by the same aminophospholipid harboring virus.

Preferably, aminophospholipid harboring virus is Dengue virus and accordingly the subject that is at risk of suffering from antibody-dependent enhancement of infection has already been infected, at least once, by Dengue Virus.

By “inhibitor” is meant an agent that is able to reduce or to abolish the interaction between an aminophospholipid, such as PtdEth and/or PtdSer, and receptor CD300a. Said inhibitor may be able to reduce or to abolish the binding of an aminophospholipid, such as PtdEth and/or PtdSer, and receptor CD300a. Said inhibitor may also be able to reduce or abolish the expression of receptor CD300a and/or be able to reduce or abolish the activity of receptor CD300a.

Such inhibitors may be organic or inorganic substances, such as lipids, peptides, polypeptides, nucleic acids, small molecules, in isolation or in mixture with other substances. In particular, such inhibitors may be antibodies, proteins, protein variants, mimetics or peptidomimetics, antisense nucleic acids, ribozymes or small molecules.

Examples of such inhibitors include, but are not limited to, an anti-CD300a antibody, an antisense nucleic acid, a mimetic or a variant CD300a protein and/or a nucleic acid, an anti-phosphatidylserine antibody, a phosphatidylserine-binding protein such as Annexin 5, an anti-phosphatidylethanolamine antibody and/or a phosphatidylethanolamine-binding protein such as Duramycin.

According to the invention, said inhibitor is (i) a CD300a inhibitor, and/or ii) an aminophospholipid binding protein.

Preferably, said inhibitor is able to reduce or to abolish the interaction between aminophospholipid, such as PtdSer and/or PtdEth, and receptor CD300a, by at least 10, 20, 30, 40%, more preferably by at least 50, 60, 70%, and most preferably by at least 75, 80, 85, 90, 95, 96, 97, 98, 99, or 100%.

Methods that can be used in order to identify an inhibitor of an interaction between CD300a and an aminophospholipid are well-known from the person skilled in the art. Examples of such methods include, but are not limited to, infection assays using typically flow cytometry or real-time quantitative PCR, Virus pull down with ELISA and/or Cell-Binding Assay. Herein, reference to polypeptides and nucleic acid includes both the amino acid sequences and nucleic acid sequences disclosed herein and variants of said sequences.

In one embodiment the mimetic or variant CD300a may be a mimetic or variant CD300a nucleic acid.

Preferably, a mimetic or a variant CD300a is a mimetic or variant CD300 protein.

Variant proteins may be naturally occurring variants, such as splice variants, alleles and isoforms, or they may be produced by recombinant means. Variations in amino acid sequence may be introduced by substitution, deletion or insertion of one or more codons into the nucleic acid sequence encoding the protein that results in a change in the amino acid sequence of the protein. Optionally the variation is by substitution of 1, 2, 3, 4, 5, 6, 7, 8, 9, 10, 11, 12, 13, 14, 15, 16, 17, 18, 19, 20 or more amino acids with any other amino acid in the protein. Additionally or alternatively, the variation may be by addition or deletion of 1, 2, 3, 4, 5, 6, 7, 8, 9, 10, 11, 12, 13, 14, 15, 16, 17, 18, 19, 20 or more amino acids within the protein.

Variant nucleic acid sequences include sequences capable of specifically hybridizing to the sequence of SEQ ID NO: 2 under moderate or high stringency conditions. Stringent conditions or high stringency conditions may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Moderately stringent conditions may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C.

Variant proteins disclosed herein are also encompassed by the invention. In the context of the invention variant proteins include fragments of the protein.

Such “fragments” may be truncated at the N-terminus or C-terminus, or may lack internal residues, for example, when compared with a full length protein. Certain fragments lack amino acid residues that are not essential for enzymatic activity. Preferably, said fragments are at least about 10, 20, 30, 40, 50, 60, 70, 80, 90, 100, 110, 120, 130, 140, 150, 160, 170, 180, 190, 200, 210, 220, 230, 240, 250, 260, 270, 280, 290 or more amino acids in length.

Fragments of the nucleic acid sequences and variants disclosed herein are also encompassed by the invention. Such fragments may be truncated at 3′ or 5′ end, or may lack internal bases, for example, when compared with a full length nucleic acid sequence. Preferably, said fragments are at least about 10, 20, 30, 40, 50, 60, 70, 80, 90, 100, 110, 120, 150, 250, 300, 350, 400, 450, 500, 600, 700, 800 or more bases in length.

“Variant proteins” may include proteins that have at least about 50% amino acid sequence identity with a polypeptide sequence disclosed herein. Preferably, a variant protein has at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% amino acid sequence identity to a full-length polypeptide sequence or a fragment of a polypeptide sequence as disclosed herein. Amino acid sequence identity is defined as the percentage of amino acid residues in the variant sequence that are identical with the amino acid residues in the reference sequence, after aligning the sequences and introducing gaps, if necessary, to achieve the maximum percent sequence identity, and not considering any conservative substitutions as part of the sequence identity. Sequence identity may be determined over the full length of the variant sequence, the full length of the reference sequence, or both.

“Variant nucleic acid sequences” may include nucleic acid sequences that have at least about 50% amino acid sequence identity with a nucleic acid sequence disclosed herein. Preferably, a variant nucleic acid sequences will have at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% amino acid sequence identity to a full-length nucleic acid sequence or a fragment of a nucleic acid sequence as disclosed herein. Nucleic acid acid sequence identity is defined as the percentage of nucleic acids in the variant sequence that are identical with the nucleic acids in the reference sequence, after aligning the sequences and introducing gaps, if necessary, to achieve the maximum percent sequence identity, and not considering any conservative substitutions as part of the sequence identity. Sequence identity may be determined over the full length of the variant sequence, the full length of the reference sequence, or both.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, or substituted with another amino acid.

In the context of the present application, the “percentage of identity” is calculated using a global alignment (i.e. the two sequences are compared over their entire length). Methods for comparing the identity of two or more sequences are well known in the art. The [symbol] needle [symbol] program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk World Wide Web site. The percentage of identity in accordance with the invention is preferably calculated using the EMBOSS: needle (global) program with a “Gap Open” parameter equal to 10.0, a “Gap Extend” parameter equal to 0.5, and a Blosum62 matrix.

Proteins consisting of an amino acid sequence “at least 80%, 85%, 90%, 95%, 96%, 97%, 98% or 99% identical” to a reference sequence may comprise mutations such as deletions, insertions and/or substitutions compared to the reference sequence. In case of substitutions, the protein consisting of an amino acid sequence at least 80%, 85%, 90%, 95%, 96%, 97%, 98% or 99% identical to a reference sequence may correspond to a homologous sequence derived from another species than the reference sequence.

“Amino acid substitutions” may be conservative or non-conservative. Preferably, substitutions are conservative substitutions, in which one amino acid is substituted for another amino acid with similar structural and/or chemical properties. The substitution preferably corresponds to a conservative substitution as indicated in the table below.

| Conservative substitutions | Type of Amino Acid |
|---|---|
| Ala, Val, Leu, Ile, Met, Pro, Phe, | Amino acids with aliphatic |
| Trp | hydrophobic side chains |
| Ser, Tyr, Asn, Gln, Cys | Amino acids with uncharged but |
| | polar side chains |
| Asp, Glu | Amino acids with acidic side chains |
| Lys, Arg, His | Amino acids with basic side chains |
| Gly | Neutral side chain |

The term “antibody” refers to immunoglobulin molecules and immunologically active portions of immunoglobulin molecules, i.e., molecules that contain an antigen binding site which immunospecifically binds an antigen. As such, the term antibody encompasses not only whole antibody molecules, but also antibody fragments as well as variants of antibodies, including derivatives such as humanized antibodies. In natural antibodies, two heavy chains are linked to each other by disulfide bonds and each heavy chain is linked to a light chain by a disulfide bond. There are two types of light chain, lambda (λ) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non hypervariable or framework regions (FR) influence the overall domain structure and hence the combining site. Complementarity determining regions (CDRs) refer to amino acid sequences which, together, define the binding affinity and specificity of the natural Fv region of a native immunoglobulin binding-site. The light and heavy chains of an immunoglobulin each have three CDRs, designated L-CDR1, L-CDR2, L-CDR3 and H-CDR1, H-CDR2, H-CDR3, respectively. Therefore, an antigen-binding site includes six CDRs, comprising the CDR set from each of a heavy and a light chain V region.

“Framework Regions” (FRs) refer to amino acid sequences interposed between CDRs, i.e. to those portions of immunoglobulin light and heavy chain variable regions that are relatively conserved among different immunoglobulins in a single species, as defined by Kabat, et al (Sequences of Proteins of Immunological Interest (National Institutes of Health, Bethesda, Md., 1991). As used herein, a “human framework region” is a framework region that is substantially identical (about 85%, or more, in particular 90%, 95%, or 100%) to the framework region of a naturally occurring human antibody.

The term “monoclonal antibody” or “mAb” as used herein refers to an antibody molecule of a single amino acid composition, that is directed against a specific antigen and which may be produced by a single clone of B cells or hybridoma. Monoclonal antibodies may also be recombinant, i.e. produced by protein engineering.

The term “chimeric antibody” refers to an engineered antibody which comprises a VH domain and a VL domain of an antibody derived from a non-human animal, in association with a CH domain and a CL domain of another antibody, in particular a human antibody. As the non-human animal, any animal such as mouse, rat, hamster, rabbit or the like can be used. A chimeric antibody may also denote a multispecific antibody having specificity for at least two different antigens.

The term “humanized antibody” refers to antibodies in which the framework or “complementarity determining regions” (CDR) have been modified to comprise the CDR from a donor immunoglobulin of different specificity as compared to that of the parent immunoglobulin. In a preferred embodiment, a mouse CDR is grafted into the framework region of a human antibody to prepare the “humanized antibody”.

“Antibody fragments” comprise a portion of an intact antibody, preferably the antigen binding or variable region of the intact antibody. Examples of antibody fragments include Fv, Fab, F(ab′)₂, Fab′, dsFv, scFv, sc(Fv)₂, diabodies and multispecific antibodies formed from antibody fragments.

The term “Fab′” refers to an antibody fragment having a molecular weight of about 50,000 Da and antigen binding activity, which is obtained by cutting a disulfide bond of the hinge region of the F(ab′)₂.

The term “F(ab′)₂” refers to an antibody fragment having a molecular weight of about 100,000 Da and antigen binding activity, which is slightly larger than the Fab bound via a disulfide bond of the hinge region, among fragments obtained by treating IgG with a protease, pepsin.

A single chain Fv (“scFv”) polypeptide is a covalently linked VH::VL heterodimer which is usually expressed from a gene fusion including VH and VL encoding genes linked by a peptide-encoding linker. The human scFv fragment of the invention includes CDRs that are held in appropriate conformation, preferably by using gene recombination techniques. “dsFv” is a VH::VL heterodimer stabilised by a disulphide bond. Divalent and multivalent antibody fragments can form either spontaneously by association of monovalent scFvs, or can be generated by coupling monovalent scFvs by a peptide linker, such as divalent sc(Fv)₂.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

The term “gene” means a DNA sequence that codes for, or corresponds to, a particular sequence of amino acids which comprises all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993, Nature 365, 566) interactions and alters the activity of the target RNA (for a review, see Stein and Cheng, 1993, Science 261, 1004, and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop or hairpin, and/or an antisense molecule can bind such that the antisense molecule forms a loop or hairpin. Thus, the antisense molecule can be complementary to 2, 3, 4, 5, 6, 7, 8, 9, 10 or more non-contiguous substrate sequences or 2, 3, 4, 5, 6, 7, 8, 9, 10 or more non-contiguous sequence portions of an antisense molecule can be complementary to a target sequence or both (for example, see Crooke, 2000, Methods Enzymol., 313, 3-45). In addition, antisense DNA can be used to target RNA by means of DNA-RNA interactions, thereby activating RNAse H, which digests the target RNA in the duplex. The antisense oligonucleotides can comprise one or more RNAse H activating region, which is capable of activating RNAse H cleavage of a target RNA.

Upon introduction, the antisense nucleic acid enters a cellular pathway that is commonly referred to as the RNA interference (RNAi) pathway. The term “RNA interference” or “RNAi” refers to selective intracellular degradation of RNA also referred to as gene silencing. RNAi also includes translational repression by small interfering RNAs (siRNAs). RNAi can be initiated by introduction of Long double-stranded RNA (dsRNAs) or siRNAs or production of siRNAs intracellularly, eg from a plasmid or transgene, to silence the expression of one or more target genes. Alternatively RNAi occurs in cells naturally to remove foreign RNAs, eg viral RNAs. Natural RNAi proceeds via dicer directed fragmentation of precursor dsRNA which direct the degradation mechanism to other cognate RNA sequences.

In some embodiments, the antisense nucleic acid may be Long double-stranded RNAs (dsRNAs), microRNA (miRNA) and/or small interferent RNA (sRNA).

As used herein “Lona double-stranded RNA” or “dsRNA” refers to an oligoribonucleotide or polyribonucleotide, modified or unmodified, and fragments or portions thereof, of genomic or synthetic origin or derived from the expression of a vector, which may be partly or fully double stranded and which may be blunt ended or contain a 5′ and/or 3′ overhang, and also may be of a hairpin form comprising a single oligoribonucleotide which folds back upon itself to give a double stranded region. In some embodiments, the dsRNA has a size ranging from 150 bp to 3000 bp, preferably ranging from 250 bp to 2000 bp, still more preferably ranging from 300 bp to 1000 bp. In some embodiments, said dsRNA has a size of at least 150, 160, 170, 180, 190, 200, 210, 220, 230, 240, 250, 260, 270, 280, 290, 300, 310, 320, 330, 340, 350, 400, 450, 500, 550, 600, 650, 700, 750, 800, 850, 900, 950, 1000, 1500 bp. In some embodiments, said dsRNA has a size of at most 3000, 2500, 2000, 1500, 1000, 950, 900, 850, 800, 750, 700, 650, 600, 550, 500, 450, 400, 350, 300 bp.

A “small interfering RNA” or “siRNA” is a RNA duplex of nucleotides that is targeted to a gene interest. A RNA duplex refers to the structure formed by the complementary pairing between two regions of a RNA molecule. siRNA is targeted to a gene in that the nucleotide sequence of the duplex portion of the siRNA is complementary to a nucleotide sequence of the targeted gene. In some embodiments, the length of the duplex of siRNAs is ranging from 15 nucleotides to 50 nucleotides, preferably ranging from 20 nucleotides to 35 nucleotides, still more preferably ranging from 21 nucleotides to 29 nucleotides. In some embodiments, the duplex can be of at least 15, 16, 17, 18, 19, 20, 21, 22, 23, 24, 25, 26, 27, 28, 29, 30, 31, 32, 33, 34, 35, 40, 45, 50 nucleotides in length. In some embodiments, the duplex can be of at most 45, 40, 35, 34, 33, 32, 31, 30, 29, 28, 27, 26, 25, 24, 23, 22, 21, 20, 19, 18, 17, 16, 15 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, or 13 nucleotides in length. The hairpin structure can also contain 3 or 5 overhang portions. In some embodiments, the overhang is a 3′ or a 5′ overhang 0, 1, 2, 3, 4, or 5 nucleotides in length.

Injection and transfection of antisense nucleic acid into cells and organisms has been the main method of delivery. However, expression vectors may also be used to continually express antisense nucleic acid in transiently and stably transfected mammalian cells. (See for example, e.g., Brummelkamp et al., 2002, Science, 296:550-553; Paddison et al., 2002, Genes & Dev, 16:948-958).

Antisense nucleic acid may be synthesized chemically or expressed via the use of a single stranded DNA expression vector or equivalent thereof using protocols known in the art as described for example in Caruthers et al., 1992, Methods in Enzymology, 211:3-19; International PCT Publication No. WO 99/54459; Brennan et al., 1998, Biotechnol Bioeng, 61:33-45; and U.S. Pat. No. 6,001,311. In a non-limiting example, small scale syntheses are conducted on a 394 Applied Biosystems, Inc. synthesizer. Alternatively, the antisense nucleic acid molecules of the present invention can be synthesized separately and joined together post-synthetically, for example by ligation (International PCT publication No. WO 93/23569; Belton et al., 1997, Bioconjugate Chem, 8:204).

The antisense nucleic acid of the invention may be able of decreasing the expression of the targeted gene, for example the gene encoding receptor CD300a, by at least 10, 20, 30, 40%, more preferably by at least 50, 60, 70%, and most preferably by at least 75, 80, 85, 90, 95, 96, 97, 98, 99, 100%.

Methods to determine the decrease in the expression of a targeted gene by an antisense nucleic acid are known to the skilled in the art and include, without limitation to it, PCR techniques such as quantitative PCR (qPCR), such as real-time or real-time quantitative PCR (RT-PCR or RT-qPCR) and/or western blot techniques.

As used herein, a “mimetic” or “peptidomimetic” is an organic molecule that mimics some properties of peptides, preferably their binding specificity and/or physiological activity. Preferred peptidomimetics are obtained by structural modification of peptides according to the invention, preferably using unnatural amino acids, D aminoacid instead of L aminoacid, conformational restraints, isosteric replacement, cyclization, or other modifications. Other preferred modifications include without limitation, those in which one or more amide bond is replaced by a non-amide bond, and/or one or more amino acid side chain is replaced by a different chemical moiety, or one of more of the N-terminus, the C-terminus or one or more side chain is protected by a protecting group, and/or double bonds and/or cyclization and/or stereospecificity is introduced into the amino acid chain to increase rigidity and/or binding affinity.

The terms “subject”, “individual” or “host” are used interchangeably and may be, for example, a human or a non-human mammal. For example, the subject is a bat; a ferret; a rabbit; a feline (cat); a canine (dog); a primate (monkey), an equine (horse); a human, including man, woman and child.

In one embodiment, the subject is at risk of suffering from antibody-dependent enhancement of infection.

Preferably, the subject who is at risk of suffering from antibody-dependent enhancement of infection has already been infected, at least once, by the same aminophospholipid harboring virus.

Inhibitor of Interaction Between PtdSer and/or PtdEth and the CD300a Receptor

“Aminophospholipid” is a phospholipid that comprises a phosphatidylgroup and an amino group and thus excludes other phospholipids such as phosphatidylcholine.

Examples of aminophospholipids in the context of the invention are Phosphatidylserine and/or Phosphatidylethanolamine.

“Phosphatidylserine” is an aminophospholipid, wherein the phosphate group is associated to the serine amino acid and is referenced under CAS number 8002-43-5. Phosphatidylserine comprises an amino group deriving from the serine amino acid and a phospholipid thus being an aminophospholipid.

“Phosphatidylethanolamine” is an aminophospholipid, in particular it is a phosphatidylserine derivative, wherein the serine amino acid is decarboxylated. It is referenced under CAS number. Phosphatidylethanolamine comprises an amino group deriving from the decarboxylated serine amino acid and a phospholipid thus being an aminophospholipid.

“CD300a”, “CD300a receptor” and “receptor CD300a” are used herein interchangeably and refers to an immunoglobulin superfamily receptor that is expressed in myeloid cells wherein the myeloid cells are for example dendritic cells, mast cells, granulocytes and/or monocytes.

A reference sequence of the cDNA coding for full-length human CD300a is available from the GenBank database under accession number NM_007261.3 (SEQ ID NO: 2) and the representative protein sequence is available under NP_009192.2 (SEQ ID NO: 1).

Preferably, said receptor CD300a comprises or consists of the amino acid sequence SEQ ID NO: 1.

Preferably, said receptor CD300a is encoded by a nucleic acid which comprises or consists of the nucleotide sequence SEQ ID NO: 2.

CD300a is also known under the synonyms IRC1; IRC2; CLM-8; IRp60; IGSF12; CMRF35H; CMRF-35H; CMRF35-H; CMRF35H9; CMRF35-H9; IRC1/IRC2; CMRF-35-H9.

CD300a receptor has a an extra cellular immunoglobulin (Ig)V-like domain, a short intracellular tail, an ectodomain and a Ca²⁺ binding site. The cytoplasmic tail of CD300a is also called intracellular tail and contains 3 classic and one nonclassic ITIM (tyrosine-based inhibitory motif) motif.

An “immunoreceptor tyrosine-based inhibition motif” (ITIM), is a conserved sequence of amino acids that is found in the cytoplasmic tails of many inhibitory receptors of the immune system. CD300a contains three canonical and one alternative ITIM motifs in its C-terminal region. In the context of the invention, in one example, this C-terminal region is not essential for the infectivity of an aminophospholipid harboring virus.

The “Ca²⁺ binding site” is a functional site that binds Ca²⁺. In the context of the invention calcium binding is important to the activity of CD300a and for the binding of CD300a to PtdSer and/or PtdEth and thus for the binding of CD300a to the aminophospholipid harboring virus. In particular the amino acids D106 to D115 of sequence SEQ ID NO: 1 have been identified as relevant to Ca²⁺ binding. However other amino acids may be as well involved in binding of Ca²⁺. Preferably, the Ca²⁺ binding site comprises the amino acid sequence SEQ ID NO: 9 corresponding to the amino acids D106 to D115 of sequence SEQ ID NO: 1.

The “ectodomain” of CD300a extends into the extracellular space. The ectodomain of CD300a in the context of the invention is thus the domain that interacts with the aminophospholipid and which is thus responsible for attachment of the aminophospholipid harboring virus and the virus entry into cells during infection.

Preferably, the ectodomain comprises the amino acid sequence SEQ ID NO: 8 corresponding to amino acids 18 to 180 of sequence SEQ ID NO: 1.

As described above, the inhibitor of an interaction between CD300a and an aminophospholipid is able to reduce or to abolish the interaction between an aminophospholipid, such as PtdEth and/or PtdSer, and receptor CD300a. Said inhibitor may be able to reduce or to abolish the binding of an aminophospholipid, such as PtdEth and/or PtdSer, and receptor CD300a. Said inhibitor may also be able to reduce or abolish the expression of receptor CD300a and/or be able to reduce or abolish the activity of receptor CD300a.

In some embodiments, the CD300a receptor inhibitor is an anti-CD300a receptor antibody, an antisense nucleic acid, a mimetic or a variant CD300a receptor.

Preferably, said CD300a receptor inhibitor is an antisense nucleic acid, and more preferably said CD300a receptor inhibitor is a siRNA. Said antisense nucleic acid may comprise or consist of a sequence that is able to inhibit or reduce the expression of CD300a receptor of sequence SEQ ID NO: 1 or a CD300a receptor of sequence encoded by the nucleic acid SEQ ID NO 2.

Preferably, said anti-CD300a receptor antibody is for example the anti-CD300a receptor antibody MAB2640 (clone 232612, rat IgG2a as obtainable from R&D systems) and/or the anti-CD300a antibody AF2640 (polyclonal goat IgG antibody as obtainable from R&D systems). Preferably, said anti-CD300a receptor antibody is directed against the ectodomain of CD300a receptor of sequence SEQ ID NO: 8. Preferably, said anti-CD300a receptor is directed to the amino acids 18 to 180 of sequence SEQ ID NO: 1.

Preferably, said anti-CD300a receptor antibody is an antibody directed against the binding site of the CD300a receptor to phosphatidylserine and/or phosphatidylethanolamine. Preferably, said antibody directed against the binding site of the CD300a receptor to phosphatidylserine and/or phosphatidylethanolamine is directed to the Ca²⁺ binding site of the CD300a receptor. Still more preferably, said anti-CD300a receptor is directed to the amino acids D106 to D115 of sequence SEQ ID NO: 1.

In some embodiments, the phosphatidylserine binding protein may be an anti-phosphatidylserine antibody or a protein that is able to bind to the phosphatidylserine, thereby blocking the interaction between phosphatidylserine and the CD300a receptor. For example, said antibody may be the anti-phosphatidylserine antibody clone 1H6 (Upstate®).

Preferably, said anti-phosphatidylserine antibody is an antibody directed against the binding site of phosphatidylserine to the CD300a receptor.

Preferably, said phosphatidylserine binding protein is Annexin V (ANX5). Preferably, said Annexin V (ANX5) protein comprises or consists of:
a) the sequence SEQ ID NO: 3 (NCBI Reference Sequence NP_001145.1, as available on Aug. 10, 2013),
b) the sequence encoded by the nucleic acid of sequence SEQ ID NO: 4 (NCBI Reference Sequence NM_001154.3, as available on Aug. 10, 2013),
c) a sequence at least 80, 85, 90, 95, 96, 97, 98, 99% identical to the sequence of a) or b).

In some embodiments, the phosphatidylethanolamine binding protein may be an anti-phosphatidylethanolamine antibody or a protein that is able to bind to the phosphatidylethanolamine, thereby blocking the interaction between phosphatidylethanolamine and the CD300a receptor. Preferably, said anti-phosphatidylethanolamine antibody is an antibody directed against the binding site of phosphatidylethanolamine to the CD300a receptor.

Preferably, said phosphatidylethanolamine binding protein is Duramycin.

Duramycin may be selected from the group consisting of Duramycin A, Duramycin B, and/or Duramycin C.

In one embodiment, Duramycin A protein comprises or consists of the amino acid sequence SEQ ID NO: 5 (UniProtKB/Swiss-Prot. reference P36504.1, as available on Aug. 10, 2013)

In one embodiment, Duramycin B protein comprises or consists of the amino acid sequence SEQ ID NO: 10 (UniProtKB/Swiss-Prot. reference P36502.1, as available on Dec. 3, 2013). In a further embodiment, Duramycin C protein comprises or consists of the amino acid sequence SEQ ID NO: 11 (UniProtKB/Swiss-Prot. reference P36503.1, as available on Dec. 3, 2013),

In one embodiment Duramycin is Duramycin A, Duramycin B, and/or Duramycin C as defined above and/or a sequence at least 80, 85, 90, 95, 96, 97, 98, 99% identical to the sequence of SEQ ID NO: 10, 11 and/or 12.

Preferably, Duramycin is Duramycin A, wherein the Duramycin A protein preferably comprises or consists of the sequence SEQ ID NO: 5 (UniProtKB/Swiss-Prot. reference P36504.1, as available on Aug. 10, 2013) and/or a sequence at least 80, 85, 90, 95, 96, 97, 98, 99% identical to the sequence SEQ ID NO: 10.

By “variant CD300a” or “variant receptor CD300a” is meant a receptor that differs from CD300a by one or several amino acid(s). For example, said variant CD300a may differ from CD300a in that it is no longer able to bind to phosphatidylserine and/or phosphatidylethanolamine. For example, said variant CD300a may differ from CD300a in that it is no longer able to bind to phosphatidylethanolamine, such as for example a CD300a receptor of sequence SEQ ID NO: 1 carrying a mutation at position D106, D112 or D115, in particular the CD300a mutants D106A (of sequence SEQ ID NO: 14), D112A (of sequence SEQ ID NO: 15) and/or D115A (of sequence SEQ ID NO: 16). For example, said variant CD300a may differ from CD300a in that it is no longer able to bind to phosphatidylserine, such as for example a CD300a receptor of sequence SEQ ID NO: 1 carrying a mutation at position D106, D112, D115 or F56, in particular the CD300a mutants D106A (of sequence SEQ ID NO: 14), D112A (of sequence SEQ ID NO: 15), D115A (of sequence SEQ ID NO: 16), or F56A (of sequence SEQ ID NO: 13).

Methods to monitor binding, for example assays to monitor the binding between variant receptor CD300a and a aminophospholipid, so called binding assays are known to the skilled in the art.

Assays may include, but are not limited to, pull down assays, typically ELISA based assays, as described for example in Meertens, L. et al. Cell Host Microbe 2012, 12(4):544-57.

### Antiviral Compounds

In a preferred embodiment, the inhibitor used in the context of the invention is for administration in combination with at least one other antiviral compound, either sequentially or simultaneously.

Sequential administration indicates that the components are administered at different times or time points, which may nonetheless be overlapping. Simultaneous administration indicates that the components are administered at the same time, but not necessarily using the same administration route.

Examples of antiviral compounds include, but are not limited to, neuraminidase inhibitors, viral fusion inhibitors, protease inhibitors, viral DNA polymerase inhibitors, signal transduction inhibitors, reverse transcriptase inhibitors, interferons, nucleoside analogs, integrase inhibitors, thymidine kinase inhibitors, viral sugar or glycoprotein synthesis inhibitors, viral structural protein synthesis inhibitors, viral attachment and adsorption inhibitors, viral entry inhibitors and their functional analogs.

Examples of neuraminidase inhibitors include, but are not limited to, oseltamivir, zanamivir and peramivir.

Examples of viral fusion inhibitors include, but are not limited to, cyclosporine, maraviroc, enfuviritide and docosanol.

Examples of protease inhibitors include, but are not limited to, saquinavir, indinarvir, amprenavir, nelfinavir, ritonavir, tipranavir, atazanavir, darunavir, zanamivir and oseltamivir.

Examples of viral DNA polymerase inhibitors include, but are not limited to, idoxuridine, vidarabine, phosphonoacetic acid, trifluridine, acyclovir, forscarnet, ganciclovir, penciclovir, cidoclovir, famciclovir, valaciclovir and valganciclovir.

Examples of signal transduction inhibitors include, but are not limited to, resveratrol and ribavirin.

Examples of nucleoside reverse transcriptase inhibitors (NRTIs) include, but are not limited to, zidovudine (ZDV, AZT), lamivudine (3TC), stavudine (d4T), zalcitabine (ddC), didanosine (2′,3′-dideoxyinosine, ddl), abacavir (ABC), emirivine (FTC), tenofovir (TDF), delaviradine (DLV), fuzeon (T-20), indinavir (IDV), lopinavir (LPV), atazanavir, combivir (ZDV/3TC), kaletra (RTV/LPV), adefovir dipivoxil and trizivir (ZDV/3TC/ABC). Non-nucleoside reverse transcriptase inhibitors (NNRTIs) may include nevirapine, delavirdine, UC-781 (thiocarboxanilide), pyridinones, TIBO, calanolide A, capravirine and efavirenz.

Examples of viral entry inhibitors include, but are not limited to, Fuzeon (T-20), NB-2, NB-64, T-649, T-1249, SCH-C, SCH-D, PRO 140, TAK 779, TAK-220, RANTES analogs, AK602, UK-427, 857, monoclonal antibodies against relevant receptors, cyanovirin-N, clyclodextrins, carregeenans, sulfated or sulfonated polymers, mandelic acid condensation polymers, AMD-3100, and functional analogs thereof.

### Method for Inhibiting Entry of a PtdSer and/or PtdEth Harboring Virus into a Cell

The inhibitor of an interaction between CD300a and an aminophospholipid according to the invention may be used in a method of inhibiting entry of an aminophospholipid harboring virus into a cell.

Said method may be an in vitro or ex vivo method, or a method of prevention or treatment of an aminophospholipid harboring virus infection as described herein.

The invention thus provides the use of an inhibitor of an interaction between CD300a and an aminophospholipid as defined herein in an in vitro or in vivo method for inhibiting entry of an aminophospholipid harboring virus into a cell. Also provided is an inhibitor of an interaction between CD300a and an aminophospholipid as defined herein for use in an in vitro or in vivo method for inhibiting entry of an aminophospholipid harboring virus into a cell.

In some embodiments, said inhibitor of an interaction between CD300a and an aminophospholipid may be used in combination with at least one other antiviral compound as defined here above.

Said method may comprise, for example, exposing said cell and/or said aminophospholipid harboring virus to said inhibitor of an interaction between CD300a and an aminophospholipid. Where the method is an in vivo method, the method may comprise administering said inhibitor of an interaction between CD300a and an aminophospholipid to a subject, preferably a patient in need thereof.

CD300a is expressed in myeloid cells for example dendritic cells, mast cells, granulocytes and monocytes.

Therefore, in some embodiments, said cells may be myeloid cells for example dendritic cells, mast cells, granulocytes and/or monocytes. CD300a is expressed in particular in monocytes. Monocytes are sensitive to antibody-dependent enhancement (ADE).

The inhibitor of an interaction between CD300a and an aminophospholipid according to the invention is therefore useful in a method of inhibiting entry of an aminophospholipid harboring virus into a cell, wherein the subject is at risk of suffering from antibody-dependent enhancement of infection as defined above.

### Pharmaceutical Compositions

The inhibitor for use according to the invention may be formulated in a pharmaceutical composition, preferably with a pharmaceutically acceptable carrier, either alone or in combination with the at least one other antiviral compound.

Pharmaceutical compositions formulated in a manner suitable for administration to human are known to the skilled in the art. The pharmaceutical composition of the invention may comprise stabilizers, buffers, and the like.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to non-toxic solid, semi-solid or liquid filler, diluent, encapsulating material or formulation auxiliary of any type. Said pharmaceutical composition may be administered orally in the form of a suitable pharmaceutical unit dosage form. The pharmaceutical compositions may be prepared in many forms that include tablets, hard or soft gelatin capsules, aqueous solutions, suspensions, and liposomes and other slow-release formulations, such as shaped polymeric gels.

The mode of administration and dosage forms are closely related to the properties of the therapeutic agents or compositions which are desirable and efficacious for the given treatment application. Suitable dosage forms include, but are not limited to, oral, intravenous, rectal, sublingual, mucosal, nasal, ophthalmic, subcutaneous, intramuscular, transdermal, spinal, intrathecal, intra-articular, intra-arterial, sub-arachnoid, bronchial, and lymphatic administration, and other dosage forms for systemic delivery of active ingredients.

Pharmaceutical compositions may be administered by any method known in the art, including, without limitation, transdermal (passive via patch, gel, cream, ointment or iontophoretic); intravenous (bolus, infusion); subcutaneous (infusion, depot); transmucosal (buccal and sublingual, e.g., orodispersible tablets, wafers, film, and effervescent formulations; conjunctival (eyedrops); rectal (suppository, enema)); or intradermal (bolus, infusion, depot).

Oral liquid pharmaceutical compositions may be in the form of, for example, aqueous or oily suspensions, solutions, emulsions, syrups or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

Pharmaceutical compositions may also be formulated for parenteral administration (e.g., by injection, for example, bolus injection or continuous infusion) and may be presented in unit dosage form in ampoules, pre-filled syringes, small volume infusion containers or multi-dose containers with an added preservative. The pharmaceutical compositions may take such forms as suspensions, solutions, or emulsions in oily or aqueous vehicles, and may contain formulating agents such as suspending, stabilizing and/or dispersing agents. Alternatively, the pharmaceutical compositions may be in powder form, obtained by aseptic isolation of sterile solid or by lyophilization from solution, for constitution with a suitable vehicle, e.g. sterile, pyrogen-free water, before use.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the pharmaceutical composition with the softened or melted carrier(s) followed by chilling and shaping in molds.

For administration by inhalation, the pharmaceutical compositions according to the invention are conveniently delivered from an insufflator, nebulizer or a pressurized pack or other convenient means of delivering an aerosol spray. Pressurized packs may comprise suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the pharmaceutical compositions may take the form of a dry powder composition, for example, a powder mix of the pharmaceutical composition and a suitable powder base such as lactose or starch. The powder composition may be presented in unit dosage form in, for example, capsules or cartridges or, e.g., gelatin or blister packs from which the powder may be administered with the aid of an inhalator or insufflator.

For intra-nasal administration, the pharmaceutical compositions may be administered via a liquid spray, such as via a plastic bottle atomizer. Typical of these are the Mistometerg (isoproterenol inhaler-Wintrop) and the Medihaler® (isoproterenol inhaler-Riker).

For antisense nucleic acid administration, the pharmaceutical compositions may be prepared in forms that include encapsulation in liposomes, microparticles, microcapsules, lipid-based carrier systems. Non limiting examples of alternative lipid based carrier systems suitable for use in the present invention include polycationic polymer nucleic acid complexes (see, e.g. US Patent Publication No 20050222064), cyclodextrin polymer nucleic acid complexes (see, e.g. US Patent Publication No 20040087024), biodegradable poly 3 amino ester polymer nucleic acid complexes (see, e.g. US Patent Publication No 20040071654), pH sensitive liposomes (see, e.g. US Patent Publication No 20020192274), anionic liposomes (see, e.g. US Patent Publication No 20030026831), cationic liposomes (see, e.g. US Patent Publication No 20030229040), reversibly masked lipoplexes (see, e.g. US Patent Publication No 20030180950), cell type specific liposomes (see, e.g. US Patent Publication No 20030198664), microparticles containing polymeric matrices (see, e.g. US Patent Publication No 20040142475), pH sensitive lipoplexes (see, e.g. US Patent Publication No 20020192275), liposomes containing lipids derivatized with releasable hydrophilic polymers (see, e.g. US Patent Publication No 20030031704), lipid entrapped nucleic acid (see, e.g. PCT Patent Publication No WO 03/057190), lipid encapsulated nucleic acid (see, e.g. US Patent Publication No 20030129221), polycationic sterol derivative nucleic acid complexes (see, e.g. U.S. Pat. No. 6,756,054), other liposomal compositions (see, e.g. US Patent Publication No 20030035829), other microparticle compositions (see, e.g. US Patent Publication No 20030157030), poly-plexes (see, e.g. PCT Patent Publication No WO 03/066069), emulsion compositions (see, e.g. U.S. Pat. No. 6,747,014), condensed nucleic acid complexes (see, e.g. US Patent Publication No 20050123600), other polycationic nucleic acid complexes (see, e.g. US Patent Publication No 20030125281), polyvinylether nucleic acid complexes (see, e.g. US Patent Publication No 20040156909), polycyclic amidinium nucleic acid complexes (see, e.g. US Patent Publication No 20030220289), nanocapsule and microcapsule compositions (see, e.g. PCT Patent Publication No WO 02/096551), stabilized mixtures of liposomes and emulsions (see, e.g. EP1304160), porphyrin nucleic acid complexes (see, e.g. U.S. Pat. No. 6,620,805), lipid nucleic acid complexes (see, e.g. US Patent Publication No 20030203865), nucleic acid micro emulsions (see, e.g. US Patent Publication No 20050037086), and cationic lipid based compositions (see, e.g. US Patent Publication No 20050234232). One skilled in the art will appreciate that siRNA of the present invention can also be delivered as a naked siRNA molecule.

Pharmaceutical compositions may also contain other excipient such as flavorings, colorings, anti-microbial agents, or preservatives.

It will be further appreciated that the amount of the pharmaceutical compositions required for use in treatment will vary not only with the therapeutic agent selected but also with the route of administration, the nature of the condition being treated and the age and condition of the patient and will be ultimately at the discretion of the attendant physician or clinician.

### Administration and Methods of Treatment

The invention also relates to a method for preventing or treating a virus infection in an individual in need thereof comprising administering a therapeutically effective amount of an inhibitor of an interaction between CD300a and an aminophospholipid according to the invention.

In one embodiment the individual in need thereof is an individual who is at risk of suffering from antibody-dependent enhancement of infection.

By “treatment” is meant a therapeutic use (i.e. on a patient having a given disease) and by “preventing” is meant a prophylactic use (i.e. on an individual susceptible of developing a given disease). The term treatment not only includes treatment leading to complete cure of the disease, but also treatments slowing down the progression of the disease and/or prolonging the survival of the patient.

An “effective amount” refers to an amount effective, at dosages and for periods of time necessary, to achieve the desired therapeutic or prophylactic result.

A therapeutically effective amount of an inhibitor of the invention may vary according to factors such as the disease state, age, sex, and weight of the individual, and the ability of the inhibitor, to elicit a desired therapeutic result. A therapeutically effective amount encompasses an amount in which any toxic or detrimental effects of the inhibitor are outweighed by the therapeutically beneficial effects. A therapeutically effective amount also encompasses an amount sufficient to confer benefit, e.g., clinical benefit.

In the context of the present invention, “preventing a virus infection” means the prevention of an aminophospholipids harboring virus infection or entry into the host cell.

In the context of the present invention, “treating a virus infection”, means reversing, alleviating, or inhibiting a virus infection or entry into the host cell.

In the context of the invention, virus infection may be reduced by at least 30, 35, 40, 45, 50, 55, 60, 65, 70, 80, 85, 90, 91, 92, 93, 94, 95, 96, 97, 98, 99, 100%.

Methods that can be used in order to determine the inhibition of the entry of virus into a host cell are well-known from the skilled person. Examples of such methods include, but are not limited to, infection assays using typically flow cytometry or real-time quantitative PCR, Virus pull down with ELISA and/or cell-binding assays.

In some embodiments, the methods of the invention comprise the administration of an inhibitor as defined above, in combination with at least one other antiviral compound as defined above, either sequentially or simultaneously.

In another embodiment, said method comprises the administration of a pharmaceutical composition as defined above.

The administration regimen may be a systemic regimen. The mode of administration and dosage forms are closely related to the properties of the therapeutic agents or compositions which are desirable and efficacious for the given treatment application. Suitable dosage forms and routes of administration include, but are not limited to, oral, intravenous, rectal, sublingual, mucosal, nasal, ophthalmic, subcutaneous, intramuscular, transdermal, spinal, intrathecal, intra-articular, intra-arterial, sub-arachnoid, bronchial, and lymphatic administration, and/or other dosage forms and routes of administration for systemic delivery of active ingredients. In a preferred embodiment, the dosage forms are for parenteral administration.

The administration regimen may be for instance for a period of at least 5, 6, 7, 8, 9, 10, 15, 20, 25, 30, 35, 40, 45, 50, 55, 60, 65, 70, 75, 80, 85, 90, 95, 100 days.

The dose range may be between 0.1 mg/kg/day and 100 mg/kg/day. More preferably, the dose range is between 0.5 mg/kg/day and 100 mg/kg/day. Most preferably, the dose range is between 1 mg/kg/day and 80 mg/kg/day. Most preferably, the dose range is between 5 mg/kg/day and 50 mg/kg/day, or between 10 mg/kg/day and 40 mg/kg/day.

In some embodiments, the dose may be of at least 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25, 30, 35, 40, 45, 50 mg/kg/day. In some embodiments, the dose may be of at most 50, 45, 40, 35, 30, 25, 20, 25, 15, 10, 5, 1, 0.5, 0.1 mg/kg/day.

The dose range may also be between 10 to 10000 Ul/kg/day. More preferably, the dose range is between 50 to 5000 Ul/kg/day, or between 100 to 1000 Ul/kg/day.

In some embodiments, the dose may be of at least 10, 25, 50, 75, 100, 150, 200, 250, 300, 350, 400, 450, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000 Ul/kg/day. In some embodiments, the dose may be of at most 10000, 9500, 9000, 8500, 8000, 7500, 7000, 6500, 6000, 5500, 5000, 4500, 4000, 3500, 3000, 2500, 2000, 1500, 1000, 900, 800, 600, 500, 450, 400, 350, 300, 250, 200, 150, 100 Ul/kg/day.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”). Furthermore the indefinite article “a” or “an” does not exclude a plurality. The mere fact that certain measures are recited in mutually different dependent claims does not indicate that a combination of these measures cannot be used to advantage.

The invention will now be described in more detail with reference to the following figures and examples. All literature and patent documents cited herein are hereby incorporated by reference. While the invention has been illustrated and described in detail in the foregoing description, the examples are to be considered illustrative or exemplary and not restrictive.

### Sequences

SEQ ID NO: 1 shows the amino acid sequence of receptor CD300a referenced under NCBI Reference Sequence Number NP_009192.2 and referenced under Uni Prot/Swissprot entry Q9UGN4.

SEQ ID NO: 2 shows the nucleic acid sequence of the cDNA encoding receptor CD300a referenced under NCBI Reference Sequence NM_007261.3.

SEQ ID NO: 3 shows the amino acid sequence of Annexin V referenced under NCBI Reference Sequence NP_001145.1.

SEQ ID NO: 4 shows the nucleic acid sequence the cDNA encoding of Annexin V referenced under NCBI Reference Sequence NM_001154.3.

SEQ ID NO: 5 shows the amino acid sequence of Duramycin A referenced under UniProtKB/Swiss-Prot. reference P36504.1

SEQ ID NO: 6 shows the amino acid sequence of the WLRD motif.

SEQ ID NO: 7 shows the amino acid sequence of the WSRD motif.

SEQ ID NO: 8 shows the amino acid sequence of the ectodomain comprising the amino acid sequence of amino acid 18 to 180 of sequence SEQ ID NO: 1.

SEQ ID NO: 9 shows the amino acid sequence of the Ca²⁺ binding site comprising the amino acid sequence of amino acid D106 to D115 of sequence SEQ ID NO: 1.

SEQ ID NO: 10 shows the amino acid sequence of Duramycin B referenced under UniProtKB/Swiss-Prot. reference P36502.1.

SEQ ID NO: 11 shows the amino acid sequence of Duramycin C referenced under UniProtKB/Swiss-Prot. reference P36503.1

SEQ ID NO: 12 shows the nucleic acid sequence of the full ORF human CD300a encoding the receptor CD300a as referenced under SEQ ID NO: 1.

SEQ ID NO: 13 shows the amino acid of human mutant CD300a F56A.

SEQ ID NO: 14 shows the amino acid of human mutant CD300a D106A.

SEQ ID NO: 15 shows the amino acid of human mutant CD300a D112A.

SEQ ID NO: 16 shows the amino acid of human mutant CD300a D115A.

SEQ ID NO: 17 shows the amino acid sequence of amino acid 71 to 121 of hCD300a.

SEQ ID NO: 18 shows the amino acid sequence of amino acid 109 to 151 of hCD300b.

SEQ ID NO: 19 shows the amino acid sequence of amino acid 78 to 128 of hCD300c.

SEQ ID NO: 20 shows the amino acid sequence of amino acid 75 to 121 of hCD300d.

SEQ ID NO: 21 shows the amino acid sequence of amino acid 72 to 124 of hCD300e.

SEQ ID NO: 22 shows the amino acid sequence of amino acid 76 to 122 of hCD300f.

### FIGURES

FIG. 1: CD300a Enhances DV Infection.

Bar chart representing infection levels of parental cells in comparison to CD300a transduced Hek293T cells. Cell surface expression for CD300a in parental and CD300a transduced Hek293T was measured by flow cytometry using a mouse monoclonal antibody. Both cell lines were challenged with DV2 JAM strain at different multiplicity of infection (MOI). Infection levels were assessed and quantified 48 h post-infection by flow cytometry using the anti-prM monoclonal antibody 2H2 in A) or mouse mAb anti NS1 in B).

FIG. 2: CD300a Enhances DV Infection.

Bar chart representing virus titers detected in the supernatant of parental cells in comparison to CD300a transduced Hek293T cells. Supernatants were collected from infected cells 48 h post-infection and virus titers were determined by plaque assays on C6/36 cells and expressed as plaque forming unit per ml.

FIG. 3: Effect of Anti-CD300Ab on DV Infection.

Graph representing the amount of infected cells in the presence of anti-CD300Ab in comparison to an isotype Antibody. Parental and CD300a Hek293T cells were infected in the presence of serial dilutions of a goat pAb IgG to CD300a or a matched isotype before infection with DV2 JAM strain. Infection levels were assayed by flow cytometry using a mouse monoclonal antibody.

FIG. 4: CD300a Enhances DV Infection.

Bar chart representing the amount of CD300a transduced Hek293T cells infected by the four different DV serotypes in comparison to parental cells. Parental and CD300a Hek293T cells were infected with a strain from each DV serotype. Infection levels were assayed by flow cytometry using a mouse monoclonal antibody. Data are represented as mean±SD of three independent experiments performed in duplicates.

FIG. 5: CD300a Enhances Infection of Aminophospholipid Harboring Virus

Bar chart representing the amount of CD300a transduced Hek293T cells infected by different virus in comparison to parental cells. Parental and CD300a Hek293T cells were infected with West Nile IS98ST1, Yellow Fever Asibi, Tick Borne Encephalitis Langat and Herpes Simplex-1 strain. The infection levels were assayed by flow cytometry using anti-E E16, 2D12 and 4G2 for West Nile IS98ST1, Yellow Fever Asibi and Tick Borne Encephalitis Langat respectively. Detection of Herpes Simplex-1 strain was with an anti-ICP4 mAb. Data are represented as mean±SD of three independent experiments performed in duplicates.

FIG. 6: CD300a Mediates DV Internalization.

Bar chart representing the amount viral RNA levels present in the cells after infection of 293T cells stably expressing CD300a in comparison to parental cells. Parental and 293T cells, stably expressing CD300a, were incubated with DV2 JAM for two hours at 37° C. Total RNA was extracted from infected cells, and viral RNA level was determined by real-time quantitative PCR with human GAPDH as endogenous control. Results from two independent experiments are expressed as the fold difference using expression in 293T infected cells as calibrator value.

FIG. 7: CD300a Mediates DV Internalization.

Bar chart representing the amount of infected cells of CD300a transduced cells in comparison to transduced cells expressing CD300a carrying a C-terminal deletion and parental cells. Cell surface expression for CD300a in parental, WT and a C-terminal deleted (ΔC-ter) version of CD300a was measured by flow cytometry using a mouse mAb to CD300a. Cells were challenged with DV2 JAM strain at MOI 2 for 48 h. Infection levels were assessed by flow cytometry using the anti-prM monoclonal antibody 2H2. Data are represented as mean±SD of two independent experiments performed in duplicates.

FIG. 8: DV is Endocytosed Internalized Via Clathrin-Coated Vesicles.

Hek293T CD300a WT were transiently transfected with a GFP control plasmid or a GFP coupled Eps15 dominant negative mutant (95-295) and infected with DV2 JAM strain at MOI 10 for 48 h. Results are expressed as the ratio between the percentages of GFP negative infected cells compared to the percentages of GFP positive infected cells. Infection levels were assessed by flow cytometry using the anti-prM monoclonal antibody 2H2. Data are represented as mean±SD of two independent experiments performed in duplicates.

FIG. 9: DV is Endocytosed Internalized Via Clathrin-Coated Vesicles.

Bar chart representing the amount of infected cells after non targeting (NT) or Clathrin Heavy Chain (CHC) specific siRNA based gene silencing. RNAi based gene silencing in Hela MZ expressing CD300a was obtained with the transfection of control non targeting (NT) or Clathrin Heavy Chain (CHC) specific siRNA for 72 h and subsequently infected with DV2 JAM strain for 48 h. Infection levels were assessed by flow cytometry using the anti-prM monoclonal antibody 2H2. Data are represented as mean±SD of two independent experiments performed in duplicates.

FIG. 10: CD300a Directly Interacts with DV.

Western Blot analysis showing the affinity of DV2 JAM particles to CD300a-Fc and/or DC-SIGN-Fc. Western Blot analysis of DV2 JAM particles pulled down with human IgG1-Fc, NKG2D-Fc, CD300a-Fc or DC-SIGN-Fc bound to BSA saturated protein A-sepharose beads. Viruses were detected through the recognition of the viral E protein with the 4G2 mAb. Identical results were obtained in two independent experiments.

FIG. 11: CD300a Directly Interacts with DV.

Bar chart representing the amount of IgG1-Fc, NKG2D-Fc, CD300a-Fc or DC-SIGN-Fc that was bound by DV2 JAM coated particles. DV2 JAM coated particles in 96-well plates were incubated with IgG1-Fc, NKG2D-Fc, CD300a-Fc or DC-SIGN-Fc (2 μg/ml) for one hour at +4° C. Bound Fcs were detected with a HRP conjugated rabbit pAb to human IgG (1/1000) and OPD substrate. Data are represented as mean±SD of three independent experiments performed in duplicates.

FIG. 12: Anti-CD300a Antibody Inhibits the Interaction with DV.

Graph representing the amount of CD300a-Fc bound by DV2 JAM coated particles in the presence of anti-CD300Ab. Serial dilutions of a rat mAb to CD300a or its IgG2a isotype were mixed with CD300a-Fc before incubation with DV2 JAM coated particles. Residual bound Fc was detected with a HRP conjugated rabbit pAb to human IgG (1/1000) and OPD substrat. Data are represented as mean±SD of three independent experiments performed in duplicates.

FIG. 13: CD300a Recognizes and Interacts with Phosphatidylethanolamine (PtdEth) and Phosphatidylserine (PtdSer) at the Surface of DV.

Bar chart representing the amount of human IgG1-Fc, TIM3-Fc or CD300a-Fc bound by Phosphatidylcholine (PtdCho), PtdEth or PtdSer. Phosphatidylcholine (PtdCho), PtdEth or PtdSer were coated on 96-well maxisorp plates in the presence of ethanol. Wells were incubated with human IgG1-Fc, TIM3-Fc or CD300a-Fc (2 μg/ml) for one hour at +4° C.

FIG. 14: CD300a Recognizes and Interacts Directly with Phosphatidylserine (PtdSer) at the Surface of DV.

Bar chart representing the amount of TIM3-Fc, CD300a-Fc or DV-SIGN-Fc bound to DV2 JAM particles in the presence of different concentration of Annexin V, a PtdSer specific ligand. DV2 JAM particles were coated in 96 well plates and incubated for one hour at +4° C. with various concentrations of the PtdSer specific ligand Annexin V before the addition of human IgG1-Fc, TIM3-Fc, CD300a-Fc or DV-SIGN-Fc (2 μg/ml). Bound Fcs were detected with a HRP conjugated rabbit pAb to human IgG (1/1000) and OPD substrate. Data are represented as mean±SD of three independent experiments performed in duplicates.

FIG. 15: CD300a Recognizes and Interacts Directly with Phosphatidylethanolamine (PtdEth) at the Surface of DV.

Bar chart representing the amount of CD300a-Fc or DV-SIGN-Fc bound to DV2 JAM particles in the presence of different concentration of Duramycin. DV2 JAM particles were coated in 96 well plates and incubated for one hour at +4° C. with various concentrations of the PtdEth specific ligand Duramycin before the addition of human IgG1-Fc, TIM3-Fc, CD300a-Fc or DV-SIGN-Fc (2 μg/ml). Bound Fcs were detected with a HRP conjugated rabbit pAb to human IgG (1/1000) and OPD substrate. Data are represented as mean±SD of three independent experiments performed in duplicates.

FIG. 16: Effect of CD300a Mutations on DV Infection

Bar chart representing the amount of infected cells when cells are transfected with CD300a WT, D106A or D115A mutants in comparison to the parental cells. Hek293T cell surface expression of CD300a in parental and CD300a WT, D106A or D115A mutants was measured by flow cytometry using a goat CD300a pAb. Cell lines were challenged with DV2 JAM at MOI 2 for 48 h. Infection levels were assessed by flow cytometry using the anti-prM monoclonal antibody 2H2. Data are represented as mean±SD of two independent experiments performed in duplicates.

FIG. 17: CD300a Interacts Directly with DV Particles

Bar chart representing the amount of CD300a-Fc bound by DV2 JAM coated particles in the presence of EDTA. Two fold serial dilutions of EDTA (1-25 mM) were mixed with CD300a-Fc before incubation with DV2 JAM coated particles. Bound Fcs were detected with a HRP conjugated rabbit pAb to human IgG (1/1000) and OPD substrate. Data shown are mean±SD of duplicates wells and representative of three independent experiments.

FIG. 18: CD300a is the Sole Member of the CD300 Family that Enhances DV Infection.

A sequence alignment showing the Ig like V-type cores of CD300a-f. WLRD motif and WSRD are underlined. The aligned amino acid sequences of CD300a-f are disclosed under SEQ ID NO: 17 to SEQ ID NO: 22, respectively.

FIG. 19: CD300a is the Sole Member of the CD300 Family that Enhances DV Infection.

Bar chart representing infection levels of Hek293T pCDNA3.1 transfected control cells in comparison to CD300a, CD300c, CD300e Hek293T expressing cells. Hek293T cells were transiently transfected with pCDNA3.1 control or with plasmid containing cDNA of CD300a, CD300c, or CD300e with lipofectamine LTX according to manufacturer instructions (Top). 18 h after transfection, cells were stained for surface expression of CD300 molecules with respective antibodies and matched isotpyes as controls (grey shading) or were infected with DV2 JAM at different MOI for 48 h (Bottom). Data are mean±SD of three independent experiments performed in duplicates

FIG. 20: CD300a Interacts Directly with DV Particles

Bar chart representing the amount of IgG1-Fc CD300a-Fc was bound by DV1, DV2, DV3 and DV4 coated particles. DV1 TVP5175, DV2 NGC, DV3 PAH881 and DV4 1036 coated particles were incubated with IgG1-Fc or CD300a-Fc. Bound Fcs were detected with a HRP conjugated rabbit pAb to human IgG (1/1000) and OPD substrate. Data shown are mean±SD of duplicates wells and representative of three independent experiments.

### EXAMPLE

The following example demonstrates that DV infection is mediated by the interaction between phosphatidylserine and/or phosphatidylethanolamine at the surface of the DV viral envelope and the receptor CD300a present at the surface of the host cell, and that such interaction can be blocked, thereby inhibiting entry of DV into host cells and preventing DV infection.

Furthermore, the example demonstrates that this interaction between aminophospholipids such as phosphatidylserine (PtdSer) and/or phosphatidylethanolamine (PtdEth) and receptor CD300a is also used by other aminophospholipid harboring viruses, in particular West-Nile Virus, Yellow Fever Virus or Dengue Virus.

### Materials and Methods

cDNA Library Screening and Plasmid Constructs

Details of the arrayed library are described in Meertens, L. et al. Cell Host Microbe 2012, 12(4):544-57. Briefly, a first round of screening was performed by forward transfection with Lipofectamine LTX (Life Technologies) of 216 pools of 8 mixed cDNAs in Hek293T cells in a 24-well plate format. An equal amount of a DC-SIGN cDNA dilution (⅛ in control plasmid) was used as a positive control. Transfected cells were then infected with DV2 JAM at a multiplicity of infection (moi) of 2 and infection was assayed 48 h later by flow cytometry using the 2H2 anti-prM mAb. A second round of screening was performed as described above with single cDNAs from pools that presented a positive intracellular staining. The CD300a ORF (SEQ ID NO: 12) was cloned in the pCR2 plasmid by T/A cloning (Life Technologies) and then subcloned between the BamHI and Xhol sites in the pTRIP plasmid. All mutations were introduced into the CD300a pCR2 construct by Quick Change Site Directed Mutagenesis (Agilent), sequence verified and subcloned in the pTRIP plasmid. cDNA coding for the CD300c and CD300e in the pCMV6-XL5 and -XL5 respectively were from Origene (CliniSciences, Nanterre, France). The Eps15 Δ95-295 GFP construct is as described in Benmerah, et al. 1999. J. Cell Sci. 112:1303-1311.

### Proteins and Antibodies

The recombinant human Ig1-Fc, NKG2D-Fc, CD300a-Fc, TIM3-Fc and DC-SIGN-Fc were from R&D Systems. The CD300-Fc was from J. Kitaura (Tokyo, Japan). Antibodies to CD300a/c are: mouse mAb MEM260 (Abcys), rat mAb 232612, goat pAb AF2640 (R&D Systems). CD300a and CD300c specific antibodies are rat mAb 6-2a and mouse 1E7D, provided by J. Kitaura (Tokyo, Japan). The CD300e goat pAb is from R&D Systems. Clathrin heavy chain and β-Tubulin rabbit pAb are from Abcam. DV antibodies are mouse mAb anti NS1, anti-prM 2H2 and anti-E 4G2 mAb. WNV and YFV anti-E proteins are the E16 and the 2D12 mAbs respectively. Infection by HSV-1 was detected by an anti ICP4 mAb (Santa Cruz biotechnologies). Polyclonal rabbit anti-human IgG-HRP and the donkey anti-goat IgG-HRP conjugated were respectively from DakoCytomation and Santa Cruz biotechnologies. Both goat pAb anti-mouse IgG-RPE and donkey anti-goat IgG-A488 conjugated were from and Jackson lmmunoresearch. Annexin V and Duramycin were both from Sigma, Lyon, France.

### Cells and Viruses

Hek293T, HeLa MZ and Vero were maintained in DMEM. Medium was supplemented with 10% FBS and 1% Penicillin/Streptomycin and L-Glutamine. Hek293T and HeLa MZ cells stably expressing CD300a WT or mutants were generated using the pTRIP lentivral vectors as described in Meertens, L. et al. Cell Host Microbe 2012, 12(4):544-57. The cell populations with high surface expression of CD300a were sorted with BD FACSAria II and FACSDiva 6.1.2 software (Becton Dickinson). The DV1 TVP5175, DV2 JAM (Jamaica), DV2 NGC (New Guinea C), DV3 PAH-881, DV4 1036, WN IS-98-ST1 and YFV Asibi were propagated in AP61 cells with limited cell passages. HSV-1(F) was propagated in Vero cells. All viruses' titters were determined on Vero cells by flow cytometry analysis (FACS) and expressed as FACS Infectious Units (FIU), except for HSV-1(F) tittered by plaque assay and expressed as Plaque Forming Units (PFU).

### Flow Cytometry and Immunofluorescence

Cell surface staining was performed by following conventional protocol in the presence of 0.02% NaN₃ and 5% FBS in cold phosphate-buffered saline (PBS) (Meertens, L. et al. Cell Host Microbe 2012). Primary antibodies were diluted at 5 μg/ml. For infection assay analysis, infected cells were fixed with PBS plus 2% (v/v) paraformaldehyde (PFA) for 15 min and permeabilized with cell surface staining buffer supplemented with 0.5% (w/v) saponin, followed by staining with mouse mAb detecting DV, WNV, YFV or HSV-1. After 30 min, primary antibodies were labeled with a goat pAb anti-mouse-IgG-RPE. Acquisition was performed on a FACSCalibur with CellQuest software (Becton Dickinson) and data analyzed by using FlowJo software (Tree Star, Olten, Switzerland). For immunofluorescence, cells were cultured on Lab-Tek II-CC2 Chamber Slide (Nunc, Roskilde, Denmark) for 48 h and pre-chilled on ice. Cells were incubated with viral particles at 4° C. for 1 h. Next, cells were extensively washed with cold PBS to remove unbound particles and shifted at 37° C. for 30 min. After washing with cold PBS, cells were fixed with PBS-PFA 4% (v/v) for 20 min at 4° C. before immunostaining with 4G2 anti-E antibody under permeabilized (+saponin 0.5%) and unpermabilized conditions. Slides were mounted with DAPI containing ProLong Gold reagent (Life Technologies).

### Virus Pull Down and ELISA

Virus pull down experiments were described in Meertens, L. et al. Cell Host Microbe 2012, 12(4):544-57. All ELISA were performed on Maxisorp 96 well plates (Nunc). PBS diluted DV particles (1.106 FIU) were coated overnight at 4° C. Coated 3-sn-phosphatidyl-Choline, -Ethanolamine or -Serine (10 μg/well, Sigma, Lyon, France) were diluted in ethanol and the wells air dried. Wells were saturated with TBS 1×, 10 mM CaCl₂, 2% BSA for 2 h at 37° C. All dilutions were in TBS 1×, 10 mM CaCl₂, washes in TBS 1×, 10 mM CaCl₂, 0.5% Tween 20, and incubations, 1 h at 4° C. Annexin V or Duramycin were added previously to Fc chimera while EDTA was mixed with Fcs′. All Fcs' were 2 μg/ml. Bound Fcs' were detected with a pAb rabbit anti-human IgG-HRP and OPD substrate.

### Cell-Binding Assay

Cells (4.10⁵) were treated with 100 U heparin in binding buffer (DMEM, NaN₃ 0.05%) containing 2% BSA for 30 min at room temperature, before incubation with viruses. Cells and DV were incubated for 90 min at 4° C. and washed twice with cold binding buffer before PBS-PFA 2% fixation. Cell surface-absorbed DV particles were stained with the 4G2 mAb, and analyzed by FACS.

RNA Purification cDNA Synthesis and RTqPCR

Total RNA was extracted from infected cells, using an RNeasy Mini Kit (QIAGEN, Courtaboeuf, France) with on-column DNase digestion, and stored at −20° C. cDNA was synthesized from 500 ng total isolated RNA by random priming-reverse transcription with the SuperScript VILO cDNA Synthesis Kit (Life Technologies). Real-time quantitative PCR (qPCR) was performed using the Fast SYBR Green Master Mix Kit on an Applied Biosystems 7500 Fast real-time PCR system (Life Technologies) (Meertens, L. et al. Cell Host Microbe 2012, 12(4):544-57). The primers for viral RNA quantification targeted a conserved region in the capsid gene. Relative expression quantification was performed based on the comparative CT method, using GAPDH as endogenous reference control.

### Results and Discussion

### Ectopic Expression of Human CD300a Enhances DV Infection

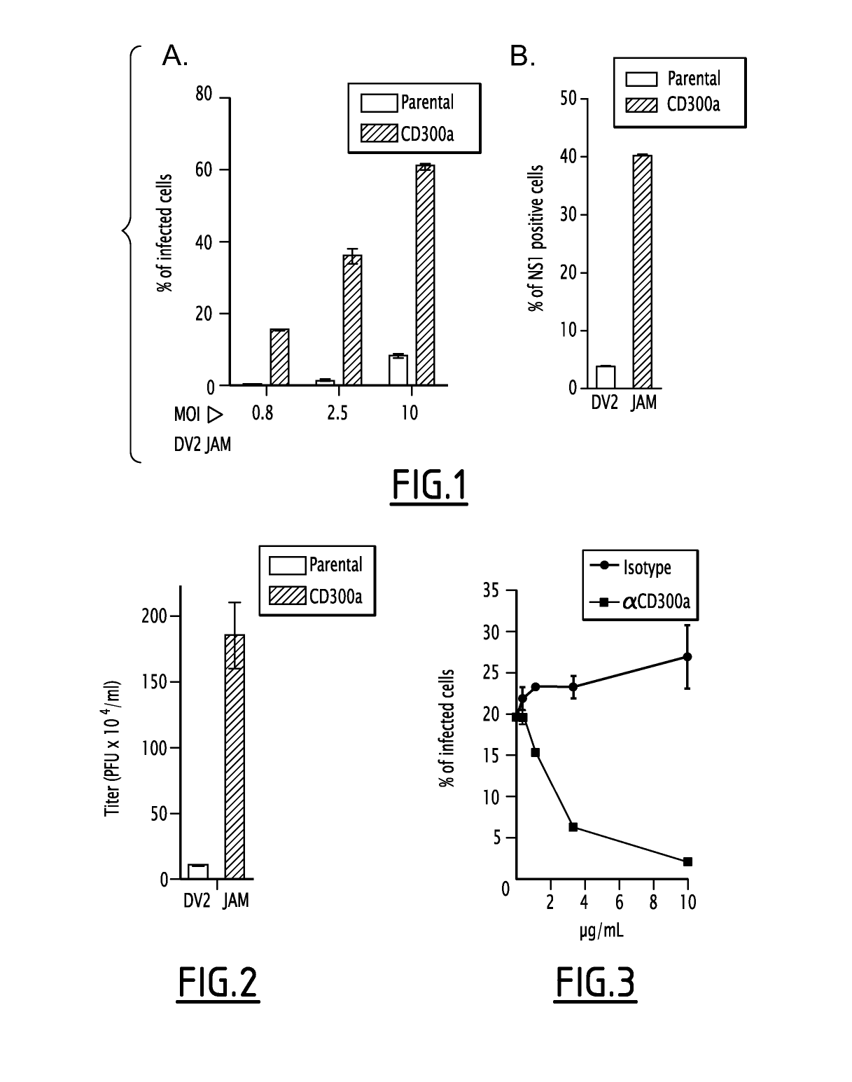

Previously TIM and TAM family members were identified by the inventors as entry factor for DV during a gain of function cDNA screen as described in Meertens, L. et al. Cell Host Microbe 2012, 12(4):544-57. Among the hits obtained during this screening, the transfection of the cDNA coding for CD300a also renders the poorly susceptible Hek293T cell line permissive to the primary, mosquito cell grown DV2 JAM strain. To confirm this result, stable CD300a expressing Hek293T cells (HekCD300a) were generated and challenged, along with the parental cells, with different multiplicity of infection (moi) of DV2 JAM. As shown in FIG. 1A, CD300a expressing cells have a twenty fold average increase of the percentage of infected cells compared to the parental cells. This result, obtained with the assessment of intracellular prM 48 h hours post infection was confirmed with the detection of the intracellular NS1 protein under the same conditions of infection (FIG. 1B). Titration of the cell free supernatants from the DV2 JAM infected HekCD300a cells showed a nearly twenty fold increase in the release of infectious virions compared to DV2 JAM infected parental cells (FIG. 2). To confirm the strict CD300a dependency of these findings, the HekCD300a cells were challenged with DV2 JAM in the presence of a goat polyclonal antibody to CD300a or a matched isotope control at concentrations ranging from 0.3 to 14.1 g/ml. Results showed in FIG. 3 present a dose dependent inhibition of the infection, reaching 80% at the highest concentration with antibody to CD300a compared to the isotype control. These results indicate that the expression of CD300a in Hek293T cells allowed a specific enhancement of infection of DV2 JAM that lead to the production of fully infectious progeny.

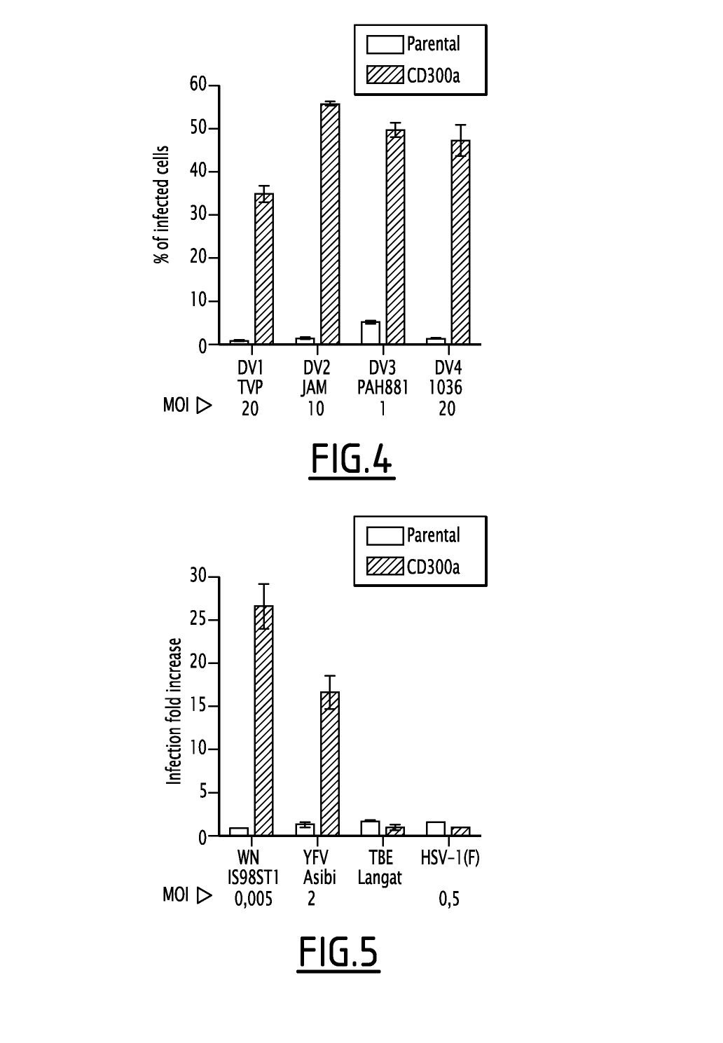

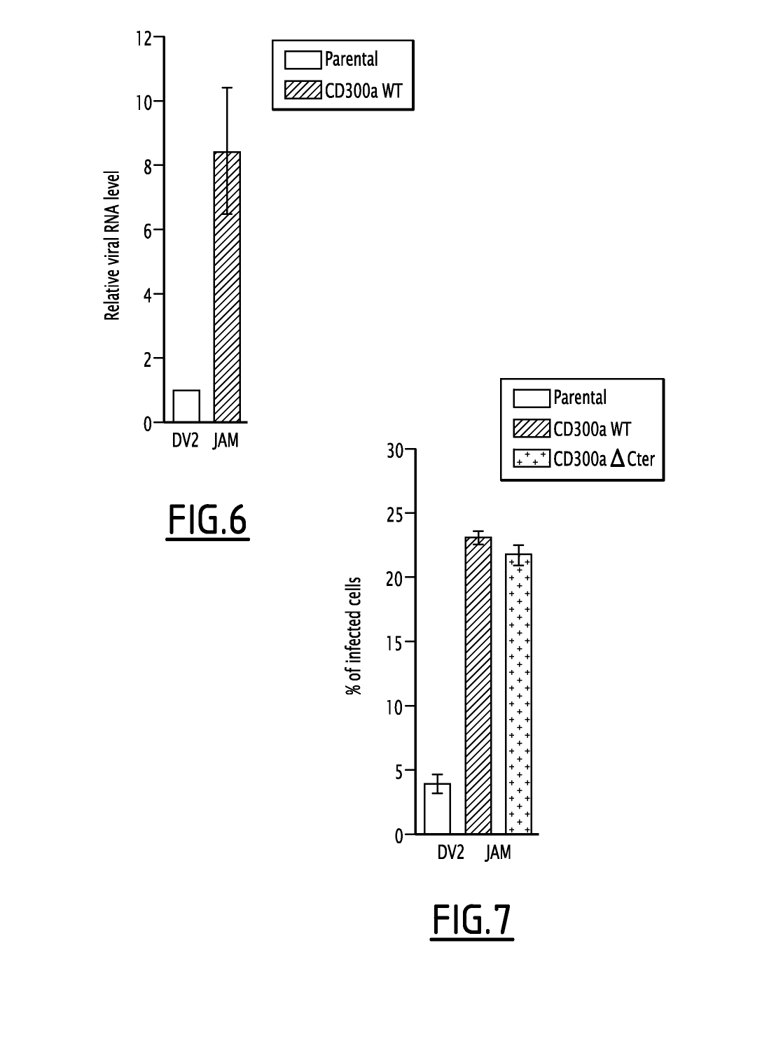

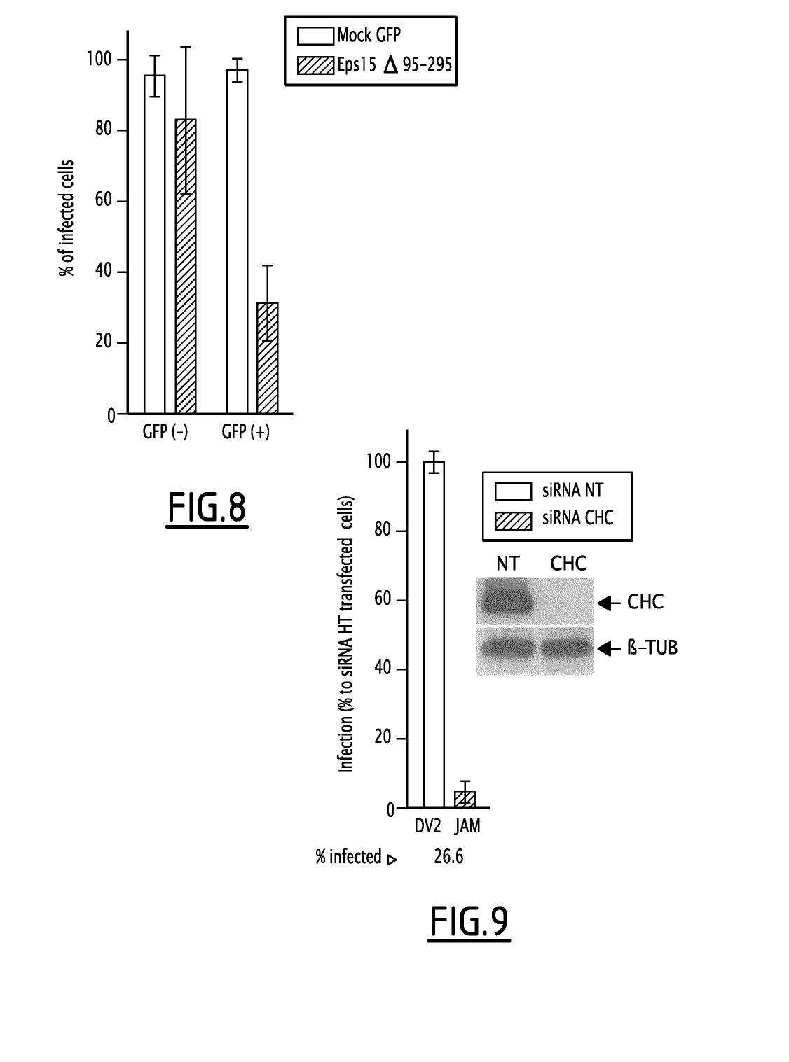

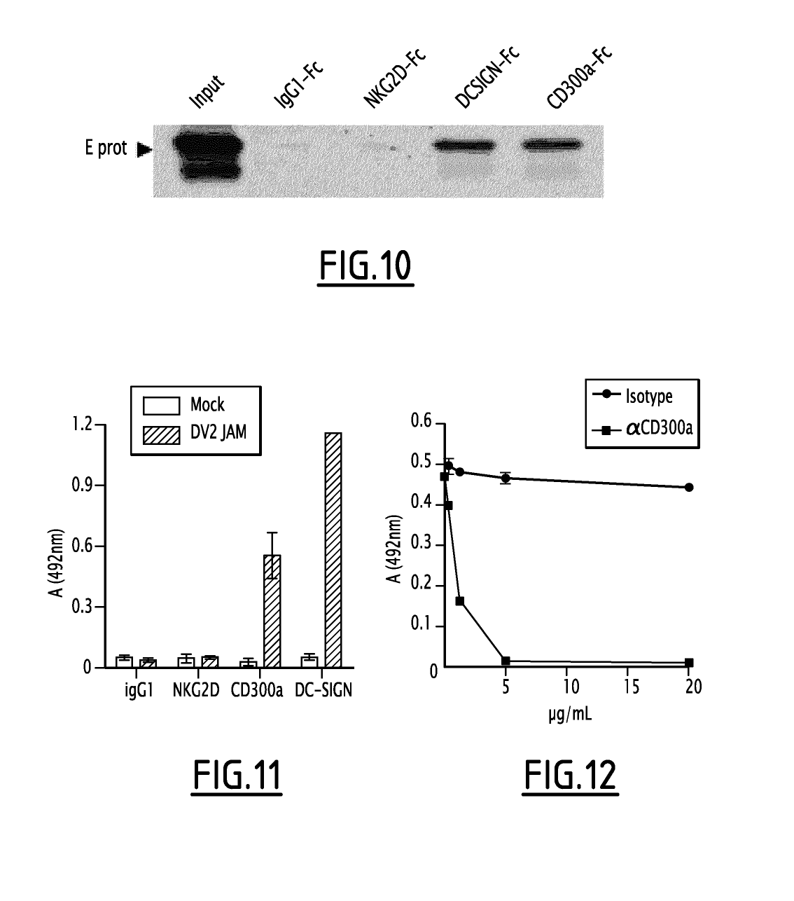

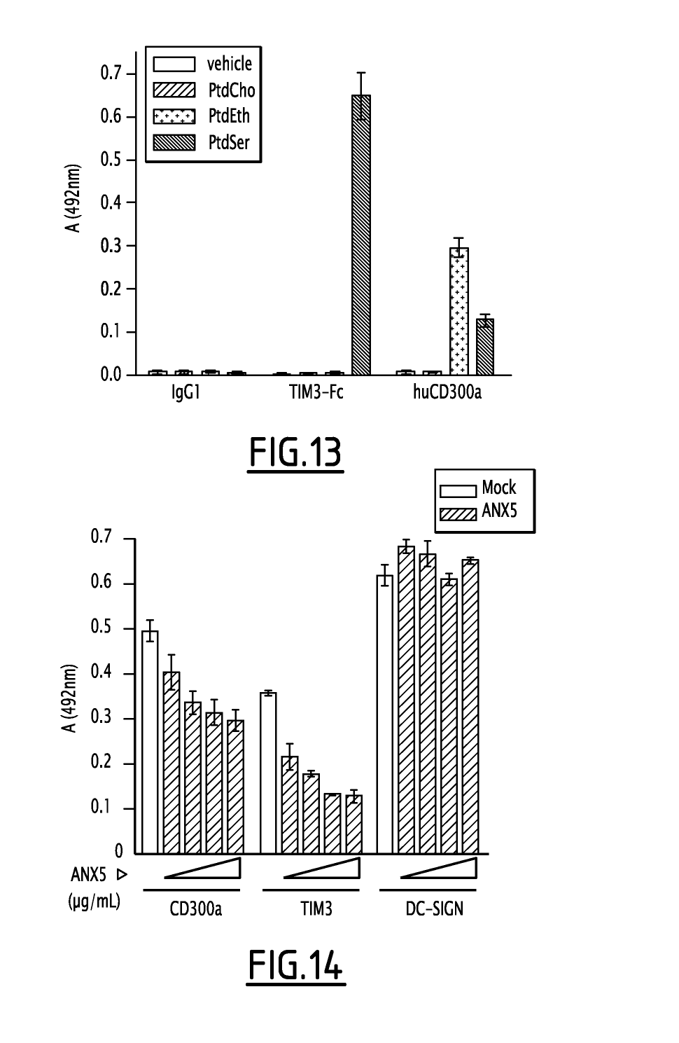

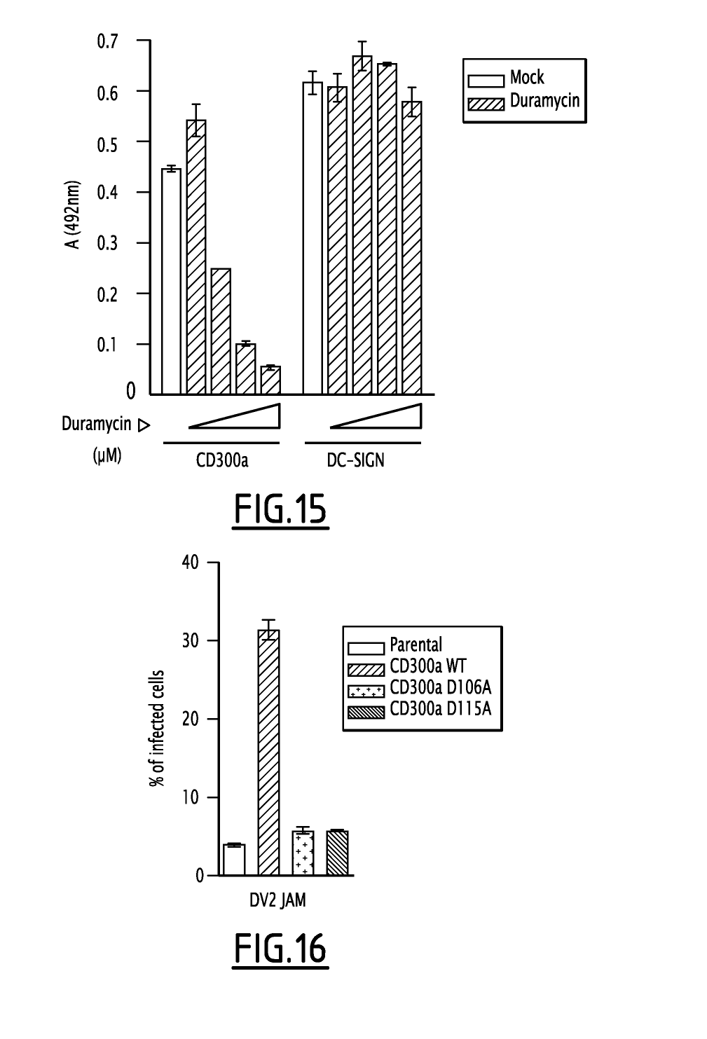

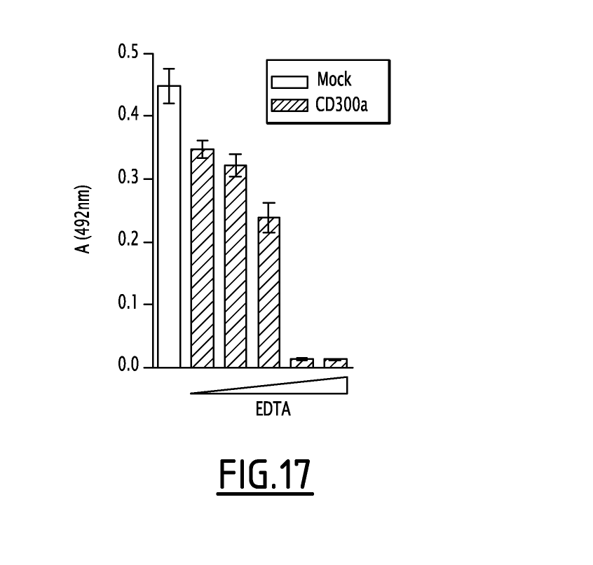

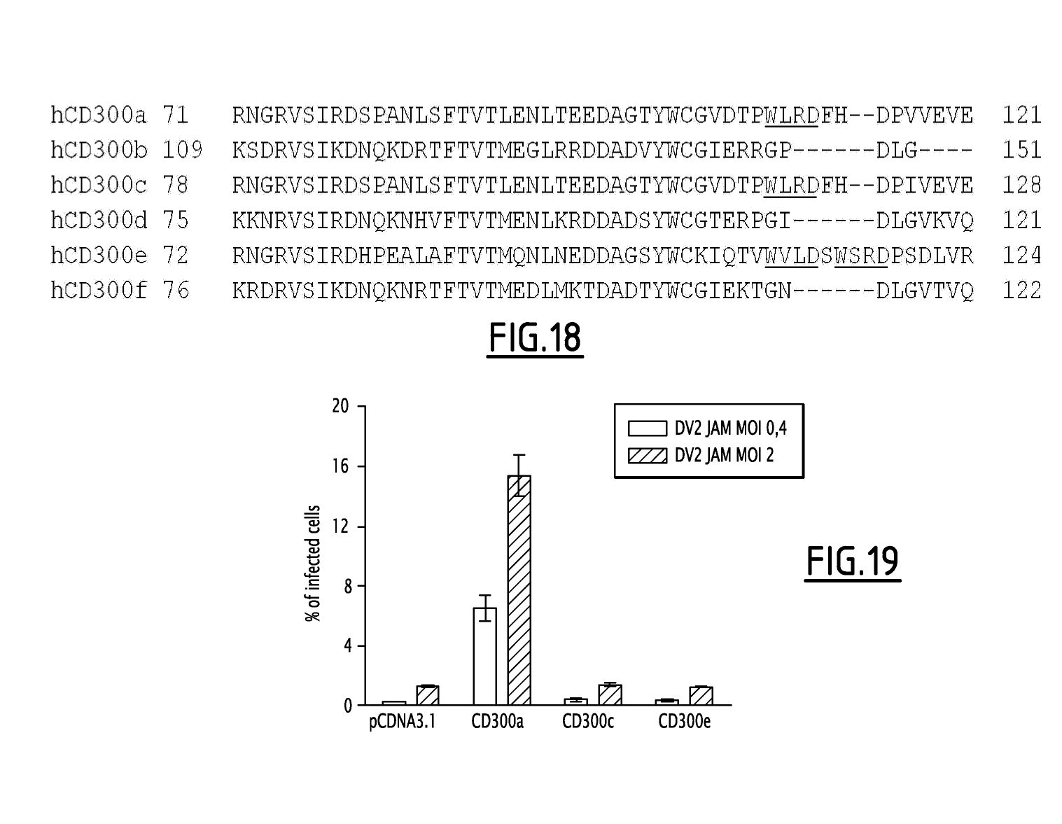

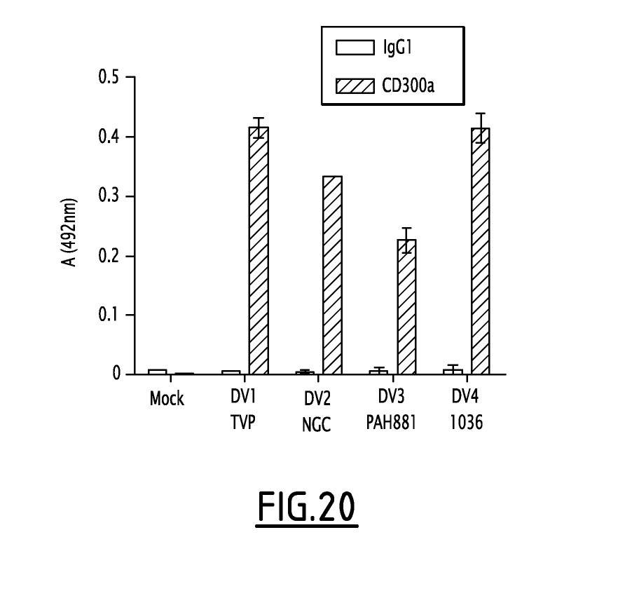

To test the possibility of a strain specific effect between CD300a and DV2 JAM, HekCD300a and parental cells were assayed with DV1 TVP5175, DV3 PAH881 and DV4 1036 primary, strains. As for DV2 JAM, CD300a ectopic expression enhanced the percentage of DV1, DV3 or DV4 infected cells within thirty, ten or forty fold respectively compared to infection in the parental cells (FIG. 4). Similar results were obtained with the primary West Nile 15-98-ST1 and YFV Asibi strains, but not with the TBE Langat strain or the herpes simplex 1 strain HSV-1(F) viruses (FIG. 5). The different behavior of TBE might be explained by the following. TBE appears to be exceptionally efficient at cleaving the protein M. Due to the efficient cleavage of Protein M the virus appears to be essentially mature and thus would largely mask the viral membrane (Junjhon, J. et aL, Journal of virology 2008, 82, 10776-10791 and Plevka, P., EMBO reports 2011, 12, 602-606). Together, these results showed that the ectopic expression of CD300a potentates the infection of all four DV subtypes strains, as well as other mosquito-born Flaviviruses.

### CD300a is an Entry Factor for DV in Ectopic Models

To characterize further the mechanism underlying the CD300a mediated enhancement of infection, the DV uptake in HekCD300a and parental cells was analyzed. DV2 JAM particles were incubated with cells at +4° C. to allow binding and then shifted to +37° C. to allow endocytosis. Intracellular uptake of viruses was detected by monitoring the E envelope glycoprotein with the 4G2 mAb in fluorescence microscopy in permeabilized and unpermeabilized conditions. Permeabilized infected HekCD300a cells presented an intracellular accumulation of DV E glycoprotein contrary to the parental cells. Surface detection of DV E glycoprotein in both cell lines was scarce, suggesting that the massive internalization proceeded only in the presence of CD300a. Similar experiments were conducted in both cell lines. Total RNA extracted and viral RNA were quantified by qPCR. The CD300a expression in Hek cells allowed a height fold increase of viral RNA uptake compared to the parental cells (FIG. 6). CD300a contains three canonical and one alternative ITIM motifs in its C-terminal region among which, two are known to be indispensable to CD300a function upon ligand binding. To ascertain the role of this domain in DV enhancement of infection, HekCD300a ΔCter cell lines were generated and one cell line was selected with comparable cell surface expression to the HekCD300a WT counterpart and further infected with DV2 JAM. Results in FIG. 7 showed that both WT and ΔCter versions of CD300a are equally effective in DV2 JAM enhancement of infection. These results indicate that CD300a mediates enhancement of infection through an increase of viral particles internalization that is likely independent of its C-terminal region.

As DV is known to be primarily internalized through the Clathrin mediated endocytosis pathway, the inventors studied the entry route of DV particles in HekCD300a that were transfected with the Eps15 Δ95-295-GFP coupled dominant negative mutant or a matched GFP control plasmid and infected them with DV2 JAM (FIG. 8). The Eps15 Δ95-295 GFP positive cells had an average 33% of infection compared to control GFP positive cells, while the GFP negative populations of both transfections presented similar average percentage of infection. To confirm the role of the Clathrin mediated pathway in the entry route of CD300a expressing cells, HeLa MZ CD300a cell line were transfected with non-targeting (NT) or Clathrin heavy chain (CHC) targeting siRNAs at a final concentration of 10 nM. The effective knockdown expression of CHC was verified by western blotting of SDS-Page separated cellular extracts (FIG. 9) and the cells were infected with DV2 JAM. CHC expression silenced cells had a nearly fifteen fold decrease of percentage of infection compared to the NT transfected cells. Collectively, these results demonstrate that the CD300a mediated uptake of DV particles route through the Clathrin mediated endocytosis. Thus, CD300a acts as an entry factor for DV in ectopic models.

CD300a Interacts Directly with DV Particles

Considering the results obtained above, it was investigated whether CD300a enhancement of infection might be based on a direct interaction with DV particles. The inventors challenged DV2 JAM particles with IgG1-, NKG2D-, CD300a- or the DC-SIGN-Fc coupled molecules diluted in 10 mM CaCl₂ TBS buffer during pull down assays with protein G-sepharose beads. Precipitated viruses were identified after SDS-Page separation through the detection of the E envelope glycoprotein by western blot analysis with the 4G2 mAb. As depicted in FIG. 10, the presence of bound DV with DC-SIGN- and CD300a-Fc, but not with NKG2D- or IgG1-Fc controls was found. The result was further confirmed by a cell based assay by incubating viral particles with parental, CD300a or DC-SIGN Hek293T cells (data not shown). Positive cell surface staining for DV particles was obtained with cells expressing CD300a or DC-SIGN. Finally, the CD300a/DV interaction was confirmed by direct ELISA based detection assay of bound Fc-coupled molecules to immobilized viral particles. As for pull down assays and in the presence of CaCl₂, the IgG1- or NKG2D-Fc control molecules did not bind to immobilized virions, while the CD300a or DC-SIGN-Fc molecules were capable to attach to DV2 JAM particles (FIG. 11), as well as DV strains from the other serotypes (FIG. 20). Of note and despite a high homology with CD300a, the CD300c-Fc molecule was unable to bind to DV (FIG. 11). Specific interaction and Ca²⁺ dependency of the interaction between DV and CD300a was further evidenced in ELISA. When CD300a-Fc molecules were incubated with a rat CD300a mAb or IgG2a isotype (ranging from 0.625 to 20 μg/ml) before the addition to DV coated wells, a concentration dependent inhibition of CD300-Fc binding to DV was observed in the presence of the CD300a mAb (FIG. 12). Similar results were obtained with EDTA (concentration range: 1.25 to 25 mM), when mixed with CD300a or DC-SIGN-Fc prior addition to DV2 JAM immobilized particles (FIG. 17). Thus CD300a binds directly and specifically to DV particles in a Ca²⁺ dependant manner.

### CD300a Recognizes and Binds to DV Envelope Derived PtdSer and PtdEth.

The inventors have previously shown that TIM family members are capable to bind directly to DV. Taking into account the PtdSer dependent nature of the interaction between TIMs and DV and the studies above showing that PtdSer is a ligand to CD300a, they investigated whether CD300a might interact with phospholipids present at the surface of DV. First an ELISA based assay was set up to confirm which phospholipids are recognized by CD300a by presenting immobilized PtdCho, PtdEth or PtdSer to soluble CD300a-Fc molecule (FIG. 13). As expected, the IgG1-Fc did not bind to any coated phospholipids, while the TIM3-Fc only interacted with PtdSer. CD300a-Fc recognized the aminophospholipids PtdSer and PtdEth but not the phospholipid PtdCho. To identify whether CD300a recognized these phospholipids at the surface of virions, DV2 JAM coated particles were challenged with serially diluted concentrations of the PtdSer specific ligand AnnexinV (ANX5) (0.31-20 μg/ml, FIG. 14) or the PtdEth specific ligand Duramycin (0.625-5 μM, FIG. 15) before the addition of Fc coupled molecules. The DV/DC-SIGN positive binding control of PtdSer or PtdEth independent nature was left unchanged in the presence of either one or the other inhibitor. Strict PtdSer dependent binding of TIM3-Fc molecule was blocked up to 60% in the presence of ANX5. Both Duramycin and ANX5 inhibited in a concentration dependent manner the binding between DV and CD300a, albeit with different efficacy. Indeed, the Duramycin inhibited up to 98%, and the AnnexinV (ANX5) inhibited up to 35% of the binding of CD300a to DV. To tie up the results of the ELISA model of interaction with the capacity of CD300a to recognize and bind to aminophospholipids at the surface of DV, the infection of Hek293T stably expressing the human CD300a single mutants D106A or D115A for which the binding to PtdEth or PtdSer is abrogated, were compared with the infection of cells expressing the WT counterpart. Results in FIG. 16 showed that even if the CD300a mutants presented higher cell surface expression than the WT, either one mutation or the other completely abolished the CD300a enhancement of infection observed with the WT. These results demonstrate that the recognition and the binding of PtdEth or PtdSer present at the surface of DV by CD300a is responsible of the gain of infection observed in the Hek293T model.

CD300a is the Sole Member of the CD300 Family that Enhances DV Infection

It was first thought that the presence of CD300a WLRD (SEQ ID NO: 6) motif, known as being indispensable to its function, would be sufficient for other CD300a members to mediate viral entry. An identical motif is present in CD300c and CD300e has a tandem alternative version WVLD (SEQ ID NO: 6)/WSRD (SEQ ID NO: 7) (FIG. 18). To the contrary, CD300b, d and f were devoid of the region bearing this motif. Hek293T cells were thus transfected with plasmids containing cDNA sequences for CD300a, CD300c or CD300e, verified cell surface expression with specific antibodies and challenged them with multiple MOI of DV2 JAM for enhancement of infection (FIG. 19). Neither CD300c nor CD300e expressing cells could promote infection above transfection control threshold, while a tenfold infection increase is observed with transient expression of CD300a. Thus, regardless to the WLRD, or related, structural signatures, CD300a seems the unique member able to potentate DV infection.

1. A method for preventing or treating a viral infection comprising administering to an individual in need hereof a therapeutically effective amount of an inhibitor of an interaction between CD300a and viral aminophospholipid.

2. The method according to claim 1, wherein
the aminophospholipid is phosphatidylserine and/or phosphatidylethanolamine.

3. The method according to claim 1, wherein the inhibitor is
(i) a CD300a inhibitor, and/or
(iii) an aminophospholipid binding protein.

4. The method according to claim 3, wherein said CD300a inhibitor is an anti-CD300a antibody, an antisense nucleic acid, a mimetic or a variant CD300a.

5. The method according to claim 3, wherein said aminophospholipid binding protein is a phosphatidylserine binding protein and/or a phosphatidylethanolamine binding protein.

6. The method according to claim 5, wherein said phosphatidylserine binding protein is an anti-phosphatidylserine antibody or Annexin 5.

7. The method according to claim 5, wherein said phosphatidylethanolamine binding protein is an anti-phosphatidylethanolamine antibody or Duramycin.

8. The method according to claim 1, wherein said virus is an aminophospholipid harboring virus.

9. The method according to claim 8, wherein said aminophospholipid harboring virus is an aminophospholipid harboring flavivirus.

10. The method according to claim 9, wherein said aminophospholipid harboring virus is a West-Nile Virus, Yellow Fever Virus or Dengue Virus.

11. The method according to claim 1, wherein said inhibitor is formulated for administration in combination with at least one other antiviral compound, either sequentially or simultaneously.

12. (canceled)
